(19) United States
(12) Patent Application Publication
(10) Pub. No.: US 2010/0167302 A1
(43) Pub. Date: Jul. 1, 2010

(54) METHOD FOR PREDICTING THE RESPONSE OF A SUBJECT SUFFERING FROM A VIRAL INFECTION OF THE LIVER TO AN ANTIVIRAL THERAPY

(75) Inventors: Witold Filipowicz, Riehen (CH); Markus Heim, Oberwil (CH); Magdalena Sarasin-Filipowicz, Basel (CH); Francois H. T. Duong, Fortschwihr (FR)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, BASEL (CH)

(21) Appl. No.: 12/676,199

(22) PCT Filed: Sep. 8, 2008
(86) PCT No.: PCT/EP08/61877

§ 371 Date: Mar. 3, 2010

(30) Foreign Application Priority Data
Sep. 10, 2007 (EP) 07116045.1

(51) Int. Cl. C12Q 1/68

(52) U.S. Cl. 435/6

(57) ABSTRACT

The application relates to treatments for improving antiviral therapies and to method for determining whether or not antiviral therapies will be effective. In particular, the present application provides a method for determining the likelihood that a subject having a viral infection of the liver will be responsive to antiviral therapy that includes stimulation of Interferon (IFN) activity, and kits for the performance of said determination.

The present invention relates to treatments for improving antiviral therapies and to method for determining whether or not antiviral therapies will be effective.

Viral infections represent a serious threat to health and are known to be a major cause of morbidity to animals and man. For instance, Hepatitis C virus (HCV) infection is a major cause of chronic liver disease worldwide. An important and striking feature of hepatitis C is its tendency towards chronicity. In over 70% of infected individuals, HCV establishes a persistent infection over decades that may lead to cirrhosis and hepatocellular carcinoma.

An interesting hypothesis in HCV biology proposes that the viral NS3-4A protease not only processes the viral proteins but also cleaves and inactivates components of the intracellular sensory pathways that detect viral infection and induce the transcriptional activation of type I interferons (IFN). One of the targets of NS3-4A is TRIF (TIR domain-containing adapter inducing IFNβ), an essential link between dsDNA detection in endosomes by TLR3 (toll-like receptor 3) and the induction of IFNβ. More recently, retinoic acid inducible gene-I (RIG-I) and MDA5 (helicard) were identified as intracellular sensors of dsRNA. Both sensors signal through Cardif (also known as IPS-1, MAVS, VISA) to induce IFNβ production. Cardif can be cleaved and inactivated by HCV NS3-4A. Two RNA helicases, RIG-I and MDA5, identified as intracellular sensors of dsRNA act through Cardif to induce IFNβ production.

Type I IFNs are not only crucial components of the innate immune system, but are also the most important components of current therapies against CHC. The current standard therapy consists of pegylated IFNα (pegIFNα) injected once weekly subcutaneously and daily intake of the oral antiviral agent ribavirin. This regimen achieves an overall sustained virological response (SVR) in about 55% of the patients, with significant differences between genotypes. An SVR is defined as the loss of detectable HCV RNA during treatment and its continued absence for at least 6 months after stopping therapy. Several studies of long-term follow-up on patients who achieve an SVR demonstrate that this response is durable in over 95% of patients. The probability of a SVR strongly depends on the early response to treatment. Patients who do not show an early virological response (EVR), defined as a decline of the viral load by at least 2 log₁₀ after 12 weeks of therapy, are highly unlikely to develop an SVR, and treatment can be stopped in these patients. On the other hand, patients with an EVR have a good chance to be cured, with 65% of them achieving a SVR. The prognosis is even better for patients who have a rapid virological response (RVR), defined as serum HCV RNA undetectable after 4 weeks of treatment. Over 85% of them will achieve a SVR. Unfortunately, less than 20% of patients with genotype 1 and about 60% of patients with genotypes 2 or 3 show a RVR. The host factors that are important for an early response to therapy are currently not known.

Type I IFNs achieve their potent antiviral effects through the regulation of hundreds of genes (ISG, interferon stimulated genes). The transcriptional activation of ISGs induces proteins that are usually not synthesized in resting cells and that establish a non-virus-specific antiviral state within the cell. Interferons induce their synthesis by activating the Jak-STAT pathway, a paradigm of cell signaling used by many cytokines and growth factors. All type I IFNs bind to the same cell surface receptor (IFNAR) and activate the receptor-associated Janus kinase family members Jak1 and Tyk2. The kinases then phosphorylate and activate signal transducer and activator of transcription 1 (STAT1) and STAT2. The activated STATs translocate into the nucleus where they bind specific DNA elements in the promoters of ISGs. Many of the ISGs have antiviral activity but others are involved in lipid metabolism, apoptosis, protein degradation and inflammatory cell responses. As is the case with many viruses, HCV interferes with the IFN system, probably at multiple levels. IFN induced Jak-STAT signaling is inhibited in cells and transgenic mice that express HCV proteins, and in liver biopsies of patients with CHC. In vitro, HCV proteins NS5A and E2 bind and inactivate protein kinase R (PKR), an important non-specific antiviral protein. However, the molecular mechanisms that are important for the response to therapeutically applied IFN in patients with CHC are currently unknown.

The capacity of HCV to interfere with the IFN pathway at many different levels is a likely mechanism underlying HCV success to establish a chronic infection (2). However, quite paradoxically, in chimpanzees acutely or chronically infected with HCV hundreds of ISGs are induced in the liver (16, 17). Nevertheless, despite the activation of the endogenous IFN system, the virus is not cleared from chronically infected animals (18). The results obtained with chimpanzees are difficult to extrapolate to humans since there are some important differences in the pathobiology of HCV infection between these species. Whereas most chimpanzees acutely infected with HCV clear the virus spontaneously, infections in men mostly become chronic. On the other hand, chronically infected chimpanzees can rarely be cured with IFN, whereas more than half of the patients with CHC are successfully treated (19).

Induction of ISGs was also found in pre-treatment liver biopsies of many patients with CHC, again demonstrating that HCV infection can lead to activation of the endogenous IFN system (20). Notably, patients with pre-elevated expression of ISGs tended to respond poorly to therapy when compared to patients having low initial expression (20). The cause of this differential response to therapy is not understood.

The present invention is based upon studies in which the inventors investigated IFN induced signaling and ISG induction in paired samples of liver biopsies and peripheral blood mononuclear cells (PBMCs) of patients with chronic hepatitis before and during therapy with pegIFNα. They further correlated biochemical and molecular data with the response to treatment. Their work is set out in more detail in the accompanying Example.

The inventors established that some subjects with a viral infection of the liver are in a state of “pre-activation”, such that the IFN signalling pathway is in a state of stimulation with activated ISGs. The inventors have found that such individuals, when subsequently treated with IFN and an antiviral agent, had a poor, or no, response to the antiviral treatment. In contrast, another group of infected subjects appeared to have no prior stimulation of IFN receptors (and stimulation of ISGs) and this group responded well to the antiviral therapy (i.e. they had a rapid virological response (RVR)). Moreover it is possible to determine whether a subject would be a poor responder to treatment or have a RVR according to the expression level of a number of specific genes, some of which are ISG genes. In other words, the inventors identified a set of genes that are prognostic genetic markers, the expression levels of which predict whether a subject will respond to antiviral treatment.

This lead the inventors to realise that a method could be developed to help a clinician decide on a treatment regimen for subjects suffering from a viral infection of the liver. Gene expression from an infected individual can be compared with gene expression from a control (i.e. a subject without viral infection).

Infected subjects with altered gene expression (compared to the control) would be unlikely to benefit from the use of IFN in a treatment regimen (i.e. these individuals would not be expected to have an RVR) whereas infected subjects for whom gene expression was mostly unaltered, compared to control expression, are likely to benefit from IFN therapy and have an RVR. The inventors were surprised to make these correlations because a skilled person would expect activation of ISGs to be associated with better viral clearance and not with a subset of subjects who respond poorly to treatment.

While there have been previous studies of gene expression levels in “responder” and “non-responder” subjects with a viral infection of the liver, e.g. Chen et al (2005) Gastroenterology 128, 1437-1444, the research conducted as part of the present invention studied a very much broader set of genes in order to determine which would act as prognostic markers. Moreover, the inventors also analysed gene expression levels in samples taken before and after antiviral treatment, and used this information to identify prognostic genetic markers, while previous studies only attempted to correlate treatment outcome to gene expression levels present in samples taken before treatment. Thus the data set which lead to the identification of the prognostic genetic markers set out below are considered to be much more complete and robust than that in previous studies.

Accordingly in the first aspect of the invention there is provided a method for determining the likelihood that a subject having a viral infection of the liver will be responsive to antiviral therapy that includes stimulation of Interferon (IFN) activity, the method comprising:
(a) analysing a sample from the subject for expression of at least one gene from each of the following groups of genes:

### (i) KYNU; PAH; LOC129607; DDC; FOLH1; YBX1; BCHE; ACADL; ACSM3; NARF; SLPI; RPS5; RPL3; RPLP0; TRIM5 and HERC5;

### (ii) HTATIP2; CDK4; IF144L; and KLHDC3;

(iii) C7; IF; IF127; IFIT1; OAS2; G1P2; OAS1; IRF7; RSAD2; IF144; OAS3; SIGIRR; and IFIT2;

### (iv) RAB4A; PPP1R1A; PPM1E; ENPP2; CAP2; ADCY1; CABYR; EVI1; PTGFRN; TRIM55; and IL28RA;

(v) MME; KCNN2; SLC16A10; AMOTL1; SPP2; LRCH4; HIST1H2BG; TSPYL5; HIST1H2AC; HIST1H2BD; PHTF1; ZNF684; GSTM5; FLJ20035; FIS; PARP12; C14 or 121; PNPT1; FLJ39051; GALNTL1; OSBPL1A; LGALS3BP; TXNRD2; LOC201725, TOMM7; SRPX2; DCN; PSMAL; MICAL-L2; FLJ30046; SAMD9; ANKRD35; LOC284013; LOC402560; and LOC147646; and,
(b) comparing expression of the genes in the sample to expression of the same genes in a control sample.

One embodiment of the invention is wherein altered expression of the genes in the sample compared to expression of the same genes in the control sample indicates that the subject is not likely to be responsive to said antiviral therapy.

An alternative embodiment of the invention is wherein unaltered expression of the genes in the sample compared to expression of the same genes in the control sample indicates that the subject is likely to be responsive to said antiviral therapy.

Further information regarding each of the genes assessed in the first aspect of the invention is provided in Table 2 in the accompanying Example. In particular, we provide the Affimetrix identification number for each of the genes, thus allowing each gene to be specifically identified.

It will be appreciated that the method of the first aspect of the invention will be of great benefit to clinicians. IFN is a protein growth factor and pharmaceutical preparations containing IFN are expensive to manufacture. It is therefore very important for a clinician to be confident that IFN is being used in an appropriate and cost-effective way. Furthermore, independent of the cost, it is often desirable to eliminate a viral infection of the liver as quickly as possible. It is therefore wasting time (which could be spent utilising alternative therapies) if a clinician administers IFN and subsequently discovers that it has no beneficial effects. The method of the first aspect of the invention is therefore of great assistance to a clinician because he can identify two populations of subjects. One population will show altered expression of the genes listed above and in table 2 and will therefore not benefit from treatment with IFN. The other population, with expression of the genes listed above and in table 2 that do not significantly differ from control samples, will benefit from therapy with IFN.

In an alternative embodiment, it is considered that the expression of the genes of Table 3 (differentially expressed 4 hours after treatment) can be used in a similar way.

By “antiviral therapy” we mean any treatment regimen for reducing viral infection that involves the stimulation of IFN activity. Such a regimen may involve the use of compounds that stimulate Type I IFN activity and/or induce IFN stimulated genes (ISGs). The therapy may involve treatment with IFN per se or other IFN receptor agonists. For example the therapy may utilise pegylated IFNα (pegIFNα).

The therapy may involve the stimulation of IFN activity alone. However the inventors have found that the method according to the first aspect of the invention is particularly useful for predicting the effectiveness of an antiviral therapy that comprises the use of a combination therapy comprising a stimulator of IFN activity in conjunction with a known antiviral agent. Many antiviral agents are known to the art and the method of the invention can be used to evaluate the effectiveness of a number of different combination therapies. However the inventors have found that the method of the first aspect of the invention has particular value for predicting the effectiveness of therapy with a stimulator of IFN activity used in conjunction with the antiviral agent ribavirin.

It is most preferred that the method of the first aspect of the invention is used to predict the usefulness of pegIFNα and ribavirin as an antiviral therapy.

The method of the first aspect of the invention may be utilised to evaluate the effectiveness of treatments for a number of different viral infections of the liver, including Hepatitis B virus and Hepatitis C virus infections. It is most preferred that the method is utilised to evaluate the effectiveness of therapies for Hepatitis C Virus (HCV) infection. The inventors have found that the method of the invention is particularly useful for distinguishing between subjects that will be expected to have a rapid virological response (RVR) and those which will not (non-RVR).

Samples representative of gene expression in a subject that may be used in accordance with the present invention encompass any sample that may provide information as to genes being expressed by the subject.

Examples of suitable samples include biopsies, samples excised during surgical procedures, blood samples, urine samples, sputum samples, cerebrospinal fluid samples, and swabbed samples (such as saliva swab samples). It will be appreciated that the source of the sample will depend upon which type of viral infection the subject may have.

It is most preferred samples are from liver tissue. Liver samples have been found to be particularly instructive when the method is applied to assessing subjects with HCV infection. The inventors were surprised to find that RVR could be distinguished from non-RVR subjects by analysing gene expression from liver samples whereas peripheral blood leukocytes exhibited no significant changes in gene expression before or after exposure to IFN.

Suitable samples may include tissue sections such as histological or frozen sections. Methods by which such sections may be prepared in such a way as to be able to provide information representative of gene expression in the subject from which the section is derived will be well known to those skilled in the art, and should be selected with reference to the technique that it is intended to use when investigating gene expression.

Although the use of samples comprising a portion of tissue from the subject is contemplated, it may generally be preferred that the sample representative of gene expression comprise a suitable extract taken from such a tissue, said extract being capable of investigation to provide information regarding gene expression in the subject. Suitable protocols which may be used for the production of tissue extracts capable of providing information regarding gene expression in a subject will be well known to those skilled in the art. Preferred protocols may be selected with reference to the manner in which gene expression is to be investigated.

In the case of samples derived from liver suitable preparation steps are disclosed in 1.1.1 and 1.1.3 of the Example.

By “control sample” we mean a sample, equivalent to that from the subject, that has been derived from an individual that is not suffering from a viral infection of the liver. Although equivalent tissue or organ samples, constituting control samples, or extracts from such samples, may be used directly as the source of information regarding gene expression in the control sample, it will be appreciated, and generally be preferred, that information regarding the expression of the selected gene (or genes) in an “ideal” control sample be provided in the form of reference data. Such reference data may be provided in the form of tables indicative of gene expression in the chosen control tissue. Alternatively, the reference data may be supplied in the form of computer software containing retrievable information indicative of gene expression in the chosen control tissue. The reference data may, for example, be provided in the form of an algorithm enabling comparison of expression of at least one selected gene(s) from each groups of genes in the subject with expression of the same genes in the control tissue sample.

In the event that expression of genes listed above and in Table 2 in a control sample is to be investigated via processing of a tissue or organ sample constituting the control sample, it is preferred that such processing is conducted using the same methods used to process the sample from the subject. Such parallel processing of subject samples and control samples allows a greater degree of confidence that comparisons of gene expression in these tissues will be normalised relative to one another (since any artefacts associated with the selected method by which tissue is processed and gene expression investigated will be applied to both the subject and control samples).

The method according to the first aspect of the invention may involve the analysis of gene expression of at least one gene, selected from each of the groups of genes. The finding that altered expression of the genes listed above and in Table 2 or 3 may be used in determining the effectiveness of an antiviral therapy is surprising, since although the expression of certain genes (such as those encoding STAT1) has been linked to HCV infection, most of the genes listed above and in Table 2 had never previously been identified as being associated IFN regulated gene expression or with the likelihood of a therapy being effective for treating viral infections. Furthermore, irrespective of the association of these genes with IFN activity, it was total unexpected that increased expression of ISGs would be associated with poor response to subsequent IFN treatment.

The inventors have identified a total of 83 different genes, the expression levels of which can be prognostic markers for the outcome of antiviral therapy. These genes have been distributed into five different groups according to their function: group (i) are considered to be involved in cell metabolism; group (ii) are considered to be involved in cell cycle; group (iii) are considered to be involved in immune response; group (iv) are considered to be involved in signal transduction; group (v) are each unassigned to any particular group set out above. This distribution is shown in the method of the invention, in which the expression level of at least one gene from each of the groups of genes is assessed in order to determine the likelihood that the subject will be responsive to antiviral therapy. The inventors have further found that these subsets of the genes have particular value and can be effective for that purpose when the expression level of at least one member of each of those groups is analysed.

It is preferred that the method is based on the analysis of at least five, six, seven, eight, nine, 10, 11, 12, 13, 14, 15, 20, 25, 30, 35, 40, 45, 50, 55, 60, 65, 70, 71, 72, 73, 74, 75, 76, 77 or 78 genes listed above.

Expression of genes listed above and in Table 2 or 3 may be investigated by analysis of target molecules representative of gene expression in the sample. The presence or absence of target molecules in a sample will generally be detected using suitable probe molecules. Such detection will provide information as to gene expression, and thereby allow comparison between gene expression occurring in the subject and expression occurring in the control sample. Probes will generally be capable of binding specifically to target molecules directly or indirectly representative of gene expression. Binding of such probes may then be assessed and correlated with gene expression to allow an effective prognostic comparison between gene expression in the subject and in the control.

By “altered expression” we include where the gene expression is both elevated or reduced in the sample when compared to the control, as discussed above.

Conversely by “unaltered expression” we include where the gene expression is not elevated or reduced in the sample when compared to the control, as discussed above.

An assessment of whether a gene expression is altered or unaltered can be made using routine methods of statistical analysis.

The target molecule may be peptide or polypeptide. Preferably the amount of peptide or polypeptide is determined using a specific binding molecule, most preferably an antibody. In a preferred instance, the amount of certain target proteins present in a sample may be assessed with reference to the biological activity of the target protein in the sample. Assessment and comparison of expression in this manner is particularly suitable in the case of protein targets having enzyme activity. Suitable techniques for the measurement of the amount of a protein target present in a sample include, but are not limited to, aptamers and antibody-based techniques, such as radio-immunoassays (RIAs), enzyme-linked immunoassays (ELISAs) and Western blotting.

Nucleic acids represent preferred target molecules for assaying gene expression according to the third aspect of the invention.

It will be understood that “nucleic acids” or “nucleic acid molecules” for the purposes of the present invention refer to deoxyribonucleotide or ribonucleotide polymers in either single- or double-stranded form. Furthermore, unless the context requires otherwise, these terms should be taken to encompass known analogues of natural nucleotides that can function in a similar manner to naturally occurring nucleotides.

Furthermore it will be understood that target nucleic acids suitable for use in accordance with the invention need not comprise “full length” nucleic acids (e.g. full length gene transcripts), but need merely comprise a sufficient length to allow specific binding of probe molecules.

It is preferred that the nucleic acid target molecule is a mRNA gene transcript and artificial products of such transcripts. Preferred examples of artificial target molecules generated from gene transcripts include cDNA and cRNA, either of which may be generated using well known protocols or commercially available kits or reagents.

In a preferred embodiment of the method of the first aspect of the invention, samples may be treated to isolate RNA target molecules by a process of lysing cells taken from a suitable sample (which may be achieved using a commercially available lysis buffer such as that produced by Qiagen Ltd.) followed by centrifugation of the lysate using a commercially available nucleic acid separation column (such as the RNeasy midi spin column produced by Qiagen Ltd). Other methods for RNA extraction include variations on the phenol and guanidine isothiocyanate method of Chomczynski, P. and Sacchi, N. (1987) Analytical Biochemistry 162, 156. “Single Step Method of RNA Isolation by Acid Guanidinium Thiocyanate-Phenol-Chloroform Extraction.” RNA obtained in this manner may constitute a suitable target molecule itself, or may serve as a template for the production of target molecules representative of gene expression.

It may be preferred that RNA derived from a subject or control sample may be used as substrate for cDNA synthesis, for example using the Superscript System (Invitrogen Corp.). The resulting cDNA may then be converted to biotinylated cRNA using the BioArray RNA Transcript labelling Kit (Enzo Life Sciences Inc.) and this cRNA purified from the reaction mixture using an RNeasy mini kit (Qiagen Ltd).

mRNA, representative of gene expression, may be measured directly in a tissue derived from a subject or control sample, without the need for mRNA extraction or purification. For example, mRNA present in, and representative of gene expression in, a subject or control sample of interest may be investigated using appropriately fixed sections or biopsies of such a tissue. The use of samples of this kind may provide benefits in terms of the rapidity with which comparisons of expression can be made, as well as the relatively cheap and simple tissue processing that may be used to produce the sample. In situ hybridisation techniques represent preferred methods by which gene expression may be investigated and compared in tissue samples of this kind. Techniques for the processing of tissues of interest that maintain the availability of RNA representative of gene expression in the subject or control sample are well known to those of skill in the art.

However, techniques by which mRNAs representative of gene expression in a subject or control sample may be extracted and collected are also well known to those skilled in the art, and the inventors have found that such techniques may be advantageously employed in accordance with the present invention. Samples comprising extracted mRNA from a subject or control sample may be preferred for use in the method of the third aspect of the invention, since such extracts tend to be more readily investigated than is the case for samples comprising the original tissues. For example, suitable target molecules allowing for comparison of gene expression may comprise the total RNA isolated from a sample of tissue from the subject, or a sample of control tissue.

Furthermore, extracted RNA may be readily amplified to produce an enlarged mRNA sample capable of yielding increased information on gene expression in the subject or control sample. Suitable examples of techniques for the extraction and amplification of mRNA populations are well known, and are considered in more detail below.

By way of example, methods of isolation and purification of nucleic acids to produce nucleic acid targets suitable for use in accordance with the invention are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993).

In a preferred method, the total nucleic acid may be isolated from a given sample using, the techniques described in the Example.

In the event that it is desired to amplify the nucleic acid targets prior to investigation and comparison of gene expression it may be preferred to use a method that maintains or controls for the relative frequencies of the amplified nucleic acids in the subject or control tissue from which the sample is derived.

Suitable methods of “quantitative” amplification are well known to those of skill in the art. One well known example, quantitative PCR, involves simultaneously co-amplifying a control sequence whose quantities are known to be unchanged between control and subject samples. This provides an internal standard that may be used to calibrate the PCR reaction.

In addition to the methods outlined above, the skilled person will appreciate that any technology coupling the amplification of gene-transcript specific product to the generation of a signal may also be suitable for quantitation. A preferred example employs convenient improvements to the polymerase chain reaction (U.S. Pat. Nos. 4,683,195 and 4,683,202) that have rendered it suitable for the exact quantitation of specific mRNA transcripts by incorporating an initial reverse transcription of mRNA to cDNA. Further key improvements enable the measurement of accumulating PCR products in real-time as the reaction progresses.

In many cases it may be preferred to assess the degree of gene expression in subject or control samples using probe molecules capable of indicating the presence of target molecules (representative of one or more of the genes listed above and in Table 2) in the relevant sample.

Probes for use in the method of the invention may be selected with reference to the product (direct or indirect) of gene expression to be investigated. Examples of suitable probes include oligonucleotide probes, antibodies, aptamers, and binding proteins or small molecules having suitable specificity.

Oligonucleotide probes constitute preferred probes suitable for use in accordance with the method of the invention. The generation of suitable oligonucleotide probes is well known to those skilled in the art (Oligonucleotide synthesis: Methods and Applications, Piet Herdewijn (ed) Humana Press (2004).). Oligonucleotide and modified oligonucleotides are commercially available from numerous companies.

For the purposes of the present invention an oligonucleotide probe may be taken to comprise an oligonucleotide capable of hybridising specifically to a nucleic acid target molecule of complementary sequence through one or more types of chemical bond. Such binding may usually occur through complementary base pairing, and usually through hydrogen bond formation. Suitable oligonucleotide probes may include natural (ie., A, G, C, or T) or modified bases (7-deazaguanosine, inosine, etc.). In addition, a linkage other than a phosphodiester bond may be used to join the bases in the oligonucleotide probe(s), so long as this variation does not interfere with hybridisation of the oligonucleotide probe to its target. Thus, oligonucleotide probes suitable for use in the methods of the invention may be peptide nucleic acids in which the constituent bases are joined by peptide bonds rather than phosphodiester linkages.

The phrase “hybridising specifically to” as used herein refers to the binding, duplexing, or hybridising of an oligonucleotide probe preferentially to a particular target nucleotide sequence under stringent conditions when that sequence is present in a complex mixture (such as total cellular DNA or RNA). Preferably a probe may bind, duplex or hybridise only to the particular target molecule.

The term “stringent conditions” refers to conditions under which a probe will hybridise to its target subsequence, but minimally to other sequences. Preferably a probe may hybridise to no sequences other than its target under stringent conditions. Stringent conditions are sequence-dependent and will be different in different circumstances. Longer sequences hybridise specifically at higher temperatures.

In general, stringent conditions may be selected to be about 5° C. lower than the thermal melting point (Tm) for the specific sequence at a defined ionic strength and pH. The Tm is the temperature (under defined ionic strength, pH, and nucleic acid concentration) at which 50% of the oligonucleotide probes complementary to a target nucleic acid hybridise to the target nucleic acid at equilibrium. As the target nucleic acids will generally be present in excess, at Tm, 50% of the probes are occupied at equilibrium. By way of example, stringent conditions will be those in which the salt concentration is at least about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide.

Oligonucleotide probes may be used to detect complementary nucleic acid sequences (i.e., nucleic acid targets) in a suitable representative sample. Such complementary binding forms the basis of most techniques in which oligonucleotides may be used to detect, and thereby allow comparison of, expression of particular genes. Preferred technologies permit the parallel quantitation of the expression of multiple genes and include technologies where amplification and quantitation of species are coupled in real-time, such as the quantitative reverse transcription PCR technologies and technologies where quantitation of amplified species occurs subsequent to amplification, such as array technologies.

Array technologies involve the hybridisation of samples, representative of gene expression within the subject or control sample, with a plurality of oligonucleotide probes wherein each probe preferentially hybridises to a disclosed gene or genes. Array technologies provide for the unique identification of specific oligonucleotide sequences, for example by their physical position (e.g., a grid in a two-dimensional array as commercially provided by Affymetrix Inc.) or by association with another feature (e.g. labelled beads as commercially provided by Illumina Inc or Luminex Inc). Oligonucleotide arrays may be synthesised in situ (e.g by light directed synthesis as commercially provided by Affymetrix Inc) or pre-formed and spotted by contact or ink-jet technology (as commercially provided by Agilent or Applied Biosystems). It will be apparent to those skilled in the art that whole or partial cDNA sequences may also serve as probes for array technology (as commercially provided by Clontech).

Oligonucleotide probes may be used in blotting techniques, such as Southern blotting or northern blotting, to detect and compare gene expression (for example by means of cDNA or mRNA target molecules representative of gene expression). Techniques and reagents suitable for use in Southern or northern blotting techniques will be well known to those of skill in the art. Briefly, samples comprising DNA (in the case of Southern blotting) or RNA (in the case of northern blotting) target molecules are separated according to their ability to penetrate a gel of a material such as acrylamide or agarose. Penetration of the gel may be driven by capillary action or by the activity of an electrical field. Once separation of the target molecules has been achieved these molecules are transferred to a thin membrane (typically nylon or nitrocellulose) before being immobilized on the membrane (for example by baking or by ultraviolet radiation). Gene expression may then be detected and compared by hybridisation of oligonucleotide probes to the target molecules bound to the membrane.

In certain circumstances the use of traditional hybridisation protocols for comparing gene expression may prove problematic. For example blotting techniques may have difficulty distinguishing between two or more gene products of approximately the same molecular weight since such similarly sized products are difficult to separate using gels. Accordingly, in such circumstances it may be preferred to compare gene expression using alternative techniques, such as those described below.

Gene expression in a sample representing gene expression in a subject may be assessed with reference to global transcript levels within suitable nucleic acid samples by means of high-density oligonucleotide array technology. Such technologies make use of arrays in which oligonucleotide probes are tethered, for example by covalent attachment, to a solid support. These arrays of oligonucleotide probes immobilized on solid supports represent preferred components to be used in the methods and kits of the invention for the comparison of gene expression. Large numbers of such probes may be attached in this manner to provide arrays suitable for the comparison of expression of large numbers of genes selected from those listed above and in Table 2. Accordingly it will be recognised that such oligonucleotide arrays may be particularly preferred in embodiments of the methods of the invention where it is desired to compare expression of more than one gene selected from each of the groups of genes listed above and in Table 2.

Other suitable methodologies that may be used in the comparison of nucleic acid targets representative of gene expression include, but are not limited to, nucleic acid sequence based am plification (NASBA); or rolling circle DNA amplification (RCA).

It is usually desirable to label probes in order that they may be easily detected. Examples of detectable moieties that may be used in the labelling of probes or targets suitable for use in accordance with the invention include any composition detectable by spectroscopic, photochemical, biochemical, immunochemical, electrical, optical or chemical means. Suitable detectable moieties include various enzymes, prosthetic groups, fluorescent materials, luminescent materials, bioluminescent materials, radioactive materials and colourimetric materials. These detectable moieties are suitable for incorporation in all types of probes or targets that may be used in the methods of the invention unless indicated to the contrary.

Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, phycoerythrin, texas red, rhodamine, green fluorescent protein, and the like; an example of a luminescent material includes luminol; examples of bioluminescent materials include luciferase, luciferin, and aequorin; examples of suitable radioactive material include ¹²⁵I, ¹³¹I, ³⁵S, ³H, ¹⁴C, or ³²P; examples of suitable colorimetric materials include colloidal gold or coloured glass or plastic (e.g., polystyrene, polypropylene, latex, etc.) beads.

Means of detecting such labels are well known to the skilled person. For example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the coloured label.

In a preferred embodiment of the invention fluorescently labelled probes or targets may be scanned and fluorescence detected using a laser confocal scanner.

In the case of labelled nucleic acid probes or targets suitable labelling may take place before, during, or after hybridisation. In a preferred embodiment, nucleic acid probes or targets for use in the methods of the invention are labelled before hybridisation. Fluorescence labels are particularly preferred and, where used, quantification of the hybridisation of the nucleic acid probes to their nucleic acid targets is by quantification of fluorescence from the hybridised fluorescently labelled nucleic acid. More preferably quantitation may be from a fluorescently labelled reagent that binds a hapten incorporated into the nucleic acid.

In a preferred embodiment of the invention analysis of hybridisation may be achieved using suitable analysis software, such as the Microarray Analysis Suite (Affymetrix Inc.).

Effective quantification may be achieved using a fluorescence microscope which can be equipped with an automated stage to permit automatic scanning of the array, and which can be equipped with a data acquisition system for the automated measurement, recording and subsequent processing of the fluorescence intensity information. Suitable arrangements for such automation are conventional and well known to those skilled in the art.

In a preferred embodiment, the hybridised nucleic acids are detected by detecting one or more detectable moieties attached to the nucleic acids. The detectable moieties may be incorporated by any of a number of means well known to those of skill in the art. However, in a preferred embodiment, such moieties are simultaneously incorporated during an amplification step in the preparation of the sample nucleic acids (probes or targets). Thus, for example, polymerase chain reaction (PCR) using primers or nucleotides labelled with a detectable moiety will provide an amplification product labelled with said moiety. In a preferred embodiment, transcription amplification using a fluorescently labelled nucleotide (e.g. fluorescein-labelled UTP and/or CTP) incorporates the label into the transcribed nucleic acids.

Alternatively, a suitable detectable moiety may be added directly to the original nucleic acid sample (e.g., mRNA, polyA mRNA, cDNA, etc. from the tissue of interest) or to an amplification product after amplification of the original nucleic acid is completed. Means of attaching labels such as fluorescent labels to nucleic acids are well known to those skilled in the art and include, for example nick translation or end-labelling (e.g. with a labeled RNA) by kinasing of the nucleic acid and subsequent attachment (ligation) of a nucleic acid linker joining the sample nucleic acid to a label (such as a suitable fluorophore).

Although the method of the first aspect of the invention is most suitable for use in association with human subjects it will be appreciated that it may also be useful in determining a course of treatment of viral infection in non-human animals (e.g. horses, dogs, cattle).

An alternative method of the invention comprises a method for determining the likelihood that a subject having a viral infection of the liver will be responsive to antiviral therapy that includes stimulation of Interferon (IFN) activity, the method comprising:

(a) analysing a sample from the subject for expression of at least one gene selected from the genes listed in Table 3 below.
(b) comparing expression of the genes in the sample to expression of the same genes in a control sample;

One embodiment of the method is wherein altered expression of the genes in the sample compared to expression of the same genes in the control sample indicates that the subject is not likely to be responsive to said antiviral therapy.

An alternative embodiment of the method is wherein unaltered expression of the genes in the sample compared to expression of the same genes in the control sample indicates that the subject is likely to be responsive to said antiviral therapy.

Techniques used for performing this aspect of the invention are provided above in relation to the first aspect of the invention. While the specific genes are different, the skilled person would appreciate and be able to identify target molecules to be assessed according to this method, as well as identify specific binding agents that can be used.

The inventors expanded their work investigating the differences between infected subjects that respond well to IFN treatment with those that do not, to examine IFN Induced Jak-STAT signalling.

IFN binds to interferon receptors and activates the Jak-STAT pathway. A central event in this activation is the phosphorylation of STAT1. The inventors found that STAT1 phosphorylation was induced in most subjects when they were treated with pegIFNα2b. However there seemed to be no correlation between STAT1 phosphorylation and the responsiveness of a subject to IFN treatment in an antiviral therapy. However the inventors were surprised to find that there were differences in responders and non-responders with regards the location of STAT1 when examined in samples. STAT1 is known to translocate into the nucleus and bind as a dimer to specific response elements in the promoters of ISGs. All rapid responding subjects had an IFN induced shift in STAT1 location following treatment with pegIFNα2b. In contrast, the non-responsive subjects (i.e. those with pre-activated IFN signalling) had no detectable STAT1 shifts; rather a large proportion of hepatocytes already had appreciable nuclear staining.

Therefore according to a second aspect of the invention, there is provided a method for determining the likelihood that a subject having a viral infection of the liver will be responsive to antiviral therapy that includes stimulation of Interferon (IFN) activity, the method comprising, examining a sample from the subject to identify the subcellular location of STAT1.

As set out in the accompanying examples, the inventors have determined that the location of STAT1 in liver cells is a prognostic marker for the responsiveness of a subject to antiviral therapy that includes stimulation of Interferon (IFN) activity. In that data it is shown that a large proportion of hepatocytes in liver samples taken from non-RVR subjects (i.e. non responders to antiviral therapy) already had an appreciable nuclear staining for STAT1 prior to antiviral therapy, whereas hepatocytes in liver samples from RVR subjects only have minimal nuclear staining. This totally unexpected finding is neither disclosed nor suggested in the art.

Thus, if a majority of hepatocytes in liver samples have nuclear staining for STAT1, then that subject is likely to be non-responsive to antiviral therapy that includes stimulation of Interferon (IFN) activity. Conversely, if a minimal number of the hepatocytes in liver samples have nuclear staining for STAT1 is likely to be responsive to antiviral therapy that includes stimulation of Interferon (IFN) activity.

Preferably the sample is a liver sample. Also preferably the method examines the subcellular location of STAT1 in hepatocyte cells.

Methods for determining the location of STAT1 protein in liver samples are routine in the art. An example of such a method is standard immunohistochemistry using commercially available anti-STAT antibodies or other specific binding entities. The accompanying example provides a detailed method for determining the location of STAT1 protein in a liver sample. Preferably the STAT1 protein examined in the method of the invention is phospho-STAT1.

By “subject” we include those subjects defined above in relation to the first aspect of the invention. Preferably the subject is human.

As set out above, the present invention is based upon studies in which the inventors investigated IFN induced signaling and ISG induction in paired samples of liver biopsies and peripheral blood mononuclear cells (PBMCs) of patients with chronic hepatitis before and during therapy with pegIFNα; this is described in more detail in the accompanying Example.

The inventors established that the endogenous IFN system is constantly activated in many infected patients. Moreover, the inventors were surprised to correlate patients with a pre-activated IFN system with a poor response to IFN therapy. This finding is counter-intuitive, because one would expect that an active innate immune system would help to eliminate the virus during IFNα therapy.

The inventors analysed ISG expression in liver biopsies and further concluded that there are patients where HCV surprisingly induces (does not block) the endogenous IFN system, and there are patients where HCV does not induce (may be by cleaving TRIF and/or Cardif) the endogenous IFN system, but that this difference has no impact on the ability of HCV to maintain a chronic infection.

In patients without a pre-activation of the IFN system, the inventors found that pegIFNα2b induced within 4 hours a robust (sub-) maximal up-regulation of many ISGs in the liver. Surprisingly, such high ISG expression levels were already present in the pretreatment biopsies of patients that later did not show a rapid virological response at week 4.

It was also found that the pre-activation of the endogenous IFN system was found more frequently in liver biopsies of patients infected with HCV genotype 1 (and 4) than with genotype 2 or 3. This is intriguing because it is well known that genotype 2 and 3 infections can be cured in over 80% of the patients, compared to less than 50% of the patients with genotype 1. The inventors finding that the frequency and degree of pre-activation of the endogenous IFN system depends on the HCV genotype provides an explanation for the different treatment susceptibility of HCV genotypes.

The inventors realised that these data establish that HCV interferes with IFN signalling and thereby impairs the response to therapy. Moreover, an inhibition of IFNα signalling by HCV explains why the strong pre-activation of the endogenous IFN system does not lead to a spontaneous elimination of HCV. The inventors do not wish to be bound by any hypothesis but believe this means that IFNα would not induce an antiviral state in the hepatocytes that are infected with HCV. The up-regulation of ISGs observed in the liver of many patients would then occur only in the non-infected hepatocytes. The strong induction of ISGs found in liver biopsies is compatible with such a model, because there are more non-infected that infected hepatocytes. IFNβ production would occur in the hepatocytes infected with a virus that is not successful in cleaving Cardif and/or TRIF. Because of the HCV induced inhibition of the Jak-STAT pathway, the secreted IFNβ would not induce an antiviral state in these infected hepatocytes, but only in non-infected neighbor cells.

The inventor realised that their new understanding of the interaction between HCV and the immune system was highly relevant to the design and selection of treatment regimens for viral infections such as HCV infection. It is therefore an aim of certain embodiments of the invention to provide novel means of treating viral infections.

According to a third aspect of the present invention, there is provided a use of an agent that reduces the activation of the IFN system for the prevention or treatment of a viral infection of the liver.

According to a fourth aspect of the present invention, there is provided an agent that reduces the activation of the IFN system in the manufacture of a medicament for the prevention or treatment of a viral infection of the liver.

The inventors, as explained above and in the Example, have realised that some subjects with a viral infection have activation of the IFN system (and associated upregulation of ISGs) and this is associated with a poor response to subsequent antiviral therapy with IFN. This lead them to realise that agents according to the third or fourth aspect of the invention, which will prevent such preactiviation, are useful for reducing the activity of the IFN pathway and will effectively “prime” a subject such that they will respond better to subsequent antiviral therapies which utilise IFN. The inventors were surprised to make these correlations because a skilled person would expect increased IFN activity in a subject to be associated with better viral clearance and not with a subset of subjects who respond poorly to treatment.

It is therefore preferred that the agents are used according to the third or fourth aspects of the invention are used to treat subjects with viral infections that also have increased (relative to uninfected control subjects) activation of IFN system.

By “reduces” we mean that agent is effective for reducing the stimulation of ISGs such that the expression levels of ISGs are not significantly different to expression levels in control tissues.

The agents may be used in the treatment of a number of different viral infections of the liver, including Hepatitis B virus and Hepatitis C virus infections. It is most preferred that the agents are used to prevent or reduce Hepatitis C Virus (HCV) infection.

Examples of agents which may be used according to the invention include where the agent may bind to the IFNα polypeptide and prevent IFN functional activity, e.g. antibodies and fragments and derivatives thereof (e.g. domain antibodies or Fabs). Alternatively the agent may act as a competitive inhibitor to IFN system by acting as an antagonist at IFNα receptors (e.g. IFNAR1, IFNAR2a, b, or c). Alternatively the agent may inhibit enzymes or other molecules in the IFN pathway. Alternatively the agent may bind to mRNA encoding IFNα polypeptide in such a manner as to lead to a reduction in that mRNA and hence a reduction in IFNα polypeptide. Alternatively the agent may bind to a nucleic sequence encoding IFNα in such a manner that it leads to a reduction in the amount of transcribed mRNA encoding IFNα polypeptide. For instance the agent may bind to coding or non-coding regions of the IFNα gene or to DNA 5′ or 3′ of the IFN and thereby reduce expression of the protein.

It is preferred that the agent of the third or fourth aspect of the invention binds to IFNα polypeptide, an IFNα receptor or to a nucleic acid encoding IFNα polypeptide.

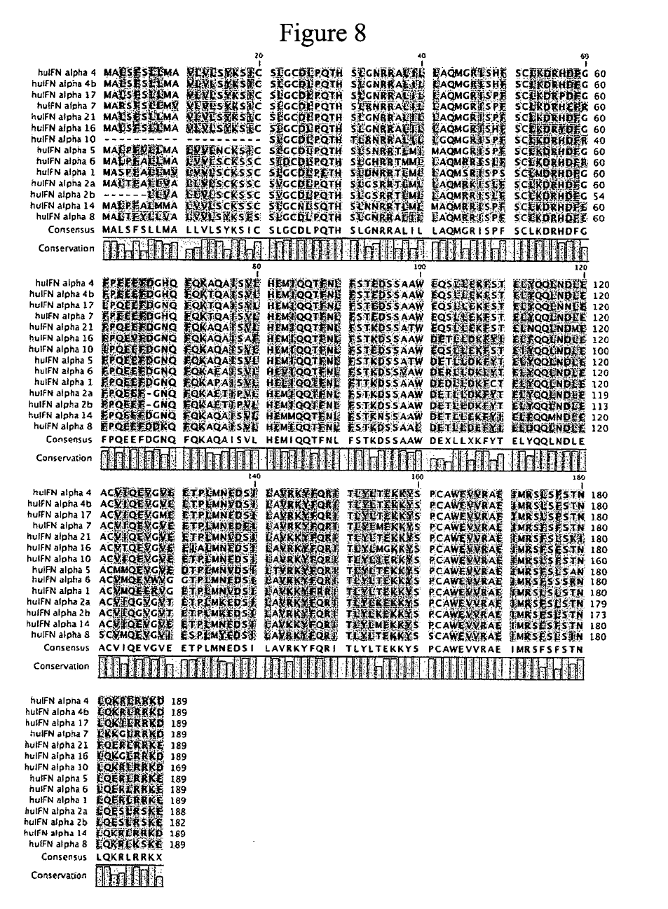

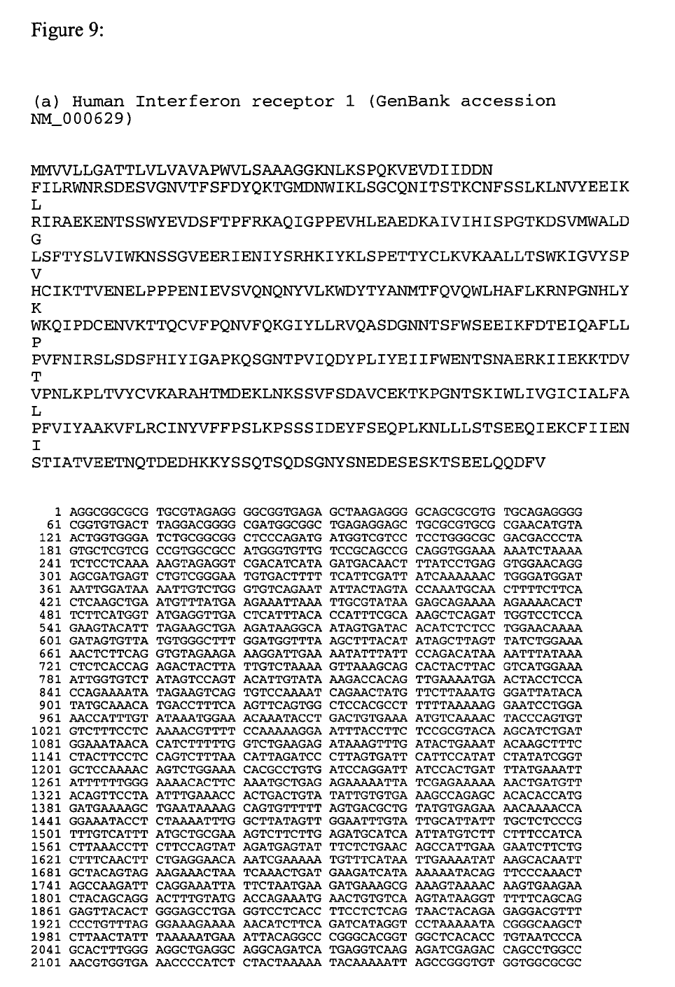

There are a number of different human Interferon α polypeptide sequences. An alignment of these sequences is shown in FIG. 8. From this alignment the following consensus sequence has been determined. This information can be used by the skilled person to develop a binding agent to IFNα polypeptide.

When the agents binds to IFNα polypeptide, it is preferred that the agent binds to an epitope defined by the protein that has been correctly folded into its native form. It will be appreciated, that there can be some sequence variability between species and also between genotypes. Accordingly other preferred epitopes will comprise equivalent regions from variants of the gene. Equivalent regions from further IFN polypeptides can be identified using sequence similarity and identity tools, and database searching methods, outlined above in the first aspect of the invention.

It is most preferred that the agent binds to a conserved region of the IFNα polypeptide or a fragment thereof. As can be seen from the alignment of IFNα polypeptide sequences in FIG. 8, there are a number of regions of amino acid sequence which are conserved between the different polypeptides. An example of such a conserved region would be positions 161 to 174 of the “consensus” sequence shown in that figure.

Agents which bind to such a region have a particularly dramatic effect on IFNα activity and are therefore particularly effective for preventing pre-activation of the IFN system and thereby improving elimination of HCV from subjects receiving antiviral therapy.

When the agents binds to an IFN receptor, it is preferred that the agent binds to and inhibits the binding of IFNα to the IFN receptor.

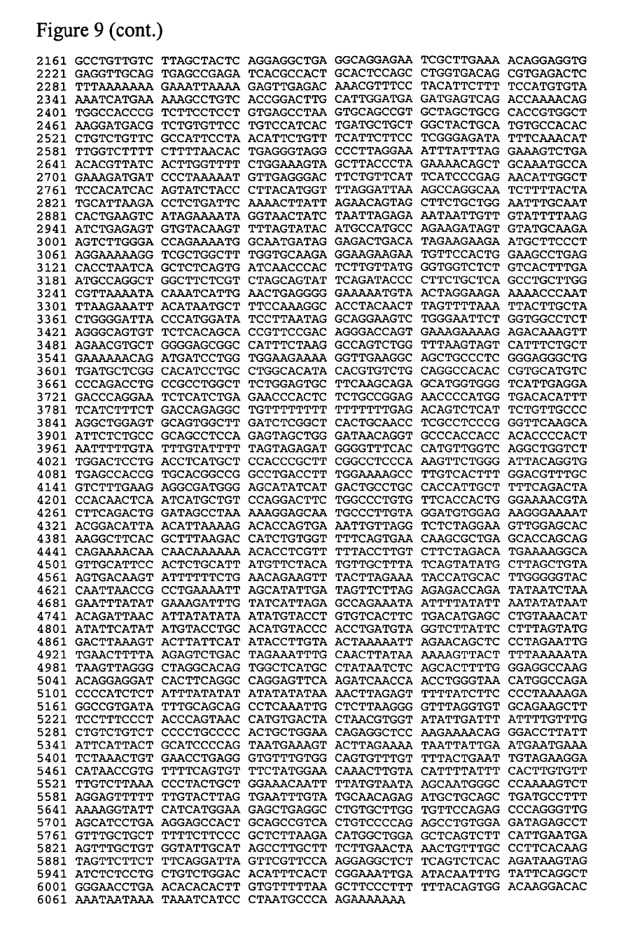

There are a number of different Interferon receptors. The amino acid sequences of these are shown in FIG. 9. This information can be used by the skilled person to develop a binding agent to IFN receptor polypeptide.

It is preferred that the agent binds to an epitope on the receptor defined by the IFN receptor protein that has been correctly folded into its native form. It will be appreciated, that there can be some sequence variability between species and also between genotypes. Accordingly other preferred epitopes will comprise equivalent regions from variants of the receptor gene. Equivalent regions from further IFN polypeptides can be identified using sequence similarity and identity tools, and database searching methods, outlined above in the first aspect of the invention.

An embodiment of the third or fourth aspects of the invention is wherein the agent is an antibody or fragment thereof.

The use of antibodies as agents to modulate polypeptide activity is well known. Indeed, therapeutic agents based on antibodies are increasingly being used in medicine. As set out above, the inventors realised that an antibody may be used to neutralise IFN system by binding thereto or may act as an inhibitor of an IFN receptor. It is therefore apparent that such agents have great utility as medicaments for the improving the treatment of HCV infections. Moreover, such antibodies can be used in the prognostic methods set out above in further aspects of the invention.

Antibodies, for use in treating human subjects, may be raised against:
(a) IFNα polypeptide per se or a number of peptides derived from the IFNα polypeptide, or peptides comprising amino acid sequences corresponding to those found in the IFNα polypeptide; or
(b) the IFN receptor or a number of peptides derived from the IFN receptor, or peptides comprising amino acid sequences corresponding to those found in the IFN receptor.

It is preferred that the antibodies are raised against antigenic structures from human IFNα polypeptide, the human IFN receptor and peptide derivatives and fragments thereof.

Antibodies may be produced as polyclonal sera by injecting antigen into animals. Preferred polyclonal antibodies may be raised by inoculating an animal (e.g. a rabbit) with antigen (e.g. all or a fragment of the IFNα polypeptide) using techniques known to the art.

Alternatively the antibody may be monoclonal. Conventional hybridoma techniques may be used to raise such antibodies. The antigen used to generate monoclonal antibodies for use in the present invention may be the same as would be used to generate polyclonal sera.

In their simplest form, antibodies or immunoglobulin proteins are Y-shaped molecules usually exemplified by the γ-immunoglobulin (IgG) class of antibodies. The molecule consists of four polypeptide chains two identical heavy (H) chains and two identical (L) chains of approximately 50 kD and 25 kD each respectively. Each light chain is bound to a heavy chain (H-L) by disulphide and non-covalent bonds. Two identical H-L chain combinations are linked to each other by similar non-covalent and disulphide bonds between the two H chains to form the basic four chain immunoglobulin structure (H-L)₂.

Light chain immunoglobulins are made up of one V-domain (V_{L}) and one constant domain (C_{L}) whereas heavy chains consist of one V-domain and, depending on H chain isotype, three or four C-domains (C_{H}1, C_{H}2, C_{H}3 and C_{H}4).

At the N-terminal region of each light or heavy chain is a variable (V) domain that varies greatly in sequence, and is responsible for specific binding to antigen. Antibody specificity for antigen is actually determined by amino acid sequences within the V-regions known as hypervariable loops or Complementarity Determining Regions (CDRs). Each H and L chain V regions possess 3 such CDRs, and it is the combination of all 6 that forms the antibody's antigen binding site. The remaining V-region amino acids which exhibit less variation and which support the hypervariable loops are called frameworks regions (FRs).

The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies for use in the invention may have very different C_{H} and C_{L} domains. Furthermore, as discussed more fully below, preferred antibody functional derivatives may comprise the Variable domains without a C-domain (e.g. scFV antibodies).

Preferred antibodies considered to be agents according to the third or fourth aspect of the invention may have the V_{L} (first domain) and V_{H} (second domain) domains. A derivative thereof may have 75% sequence identity, more preferably 90% sequence identity and most preferably has at least 95% sequence identity. It will be appreciated that most sequence variation may occur in the framework regions (FRs) whereas the sequence of the CDRs of the antibodies, and functional derivatives thereof, should be most conserved.

A number of preferred embodiments of the agent of the third or fourth aspects of the invention relate to molecules with both Variable and Const ant domains. However it will be appreciated that antibody fragments (e.g. scFV antibodies or FAbs) are also encompassed by the invention that comprise essentially the Variable region of an antibody without any Constant region.

An scFV antibody fragment considered to be an agent of the third or fourth aspect of the invention may comprise the whole of the V_{H} and V_{L} domains of an antibody raised against IFN polypeptide. The V_{H} and V_{L} domains may be separated by a suitable linker peptide.

Antibodies, and particularly mAbs, generated in one species are known to have several serious drawbacks when used to treat a different species. For instance when murine antibodies are used in humans they tend to have a short circulating half-life in serum and may be recognised as foreign proteins by the immune system of a patient being treated. This may lead to the development of an unwanted human anti-mouse antibody (HAMA) response. This is particularly troublesome when frequent administration of an antibody is required as it can enhance its clearance, block its therapeutic effect, and induce hypersensitivity reactions. These factors limit the use of mouse monoclonal antibodies in human therapy and have prompted the development of antibody engineering technology to generate humanised antibodies.

Therefore, where the antibody capable of reducing IFN activity is to be used as a therapeutic agent for treating HCV infections in a human subject, then it is preferred that antibodies and fragments thereof of non-human source are humanised.

Humanisation may be achieved by splicing V region sequences (e.g. from a monoclonal antibody generated in a non-human hybridoma) with C region (and ideally FRs from V region) sequences from human antibodies. The resulting ‘engineered’ antibodies are less immunogenic in humans than the non-human antibodies from which they were derived and so are better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric molecules may be used to treat or prevent cancer according to the present invention.

Further humanisation of antibodies may involve CDR-grafting or reshaping of antibodies. Such antibodies are produced by transplanting the heavy and light chain CDRs of a non-human antibody (which form the antibody's antigen binding site) into the corresponding framework regions of a human antibody.

Humanised antibody fragments represent preferred agents for use according to the invention. Human FAbs recognising an epitope on IFNα polypeptide or an IFN receptor may be identified through screening a phage library of variable chain human antibodies. Techniques known to the art (e.g as developed by Morphosys or Cambridge Antibody Technology) may be employed to generate Fabs that may be used as agents according to the invention. In brief a human combinatorial Fab antibody library may be generated by transferring the heavy and light chain variable regions from a single-chain Fv library into a Fab display vector. This library may yield 2.1×10¹⁰ different antibody fragments. The peptide may then be used as “bait” to identify antibody fragments from then library that have the desired binding properties.

Domain antibodies (dAbs) represent another preferred agent that may be used according to this embodiment of the invention. dAbs are the smallest functional binding unit of antibodies and correspond to the variable regions of either the heavy or light chains of human antibodies. Such dAbs may have a molecule weight of around 13 kDa (corresponding to about 1/10 (or less) the size of a full antibody).

According to another embodiment of the third and fourth aspects of the invention, peptides may be used to reduce IFNα polypeptide activity. Such peptides represent other preferred agents for use according to the invention. These peptides may be isolated, for example, from libraries of peptides by identifying which members of the library are able to reduce the activity or expression of IFN α polypeptide. Suitable libraries may be generated using phage display techniques.

Aptamers represent another preferred agent of the third or fourth aspect of the invention. Aptamers are nucleic acid molecules that assume a specific, sequence-dependent shape and bind to specific target ligands based on a lock-and-key fit between the aptamer and ligand. Typically, aptamers may comprise either single- or double-stranded DNA molecules (ssDNA or dsDNA) or single-stranded RNA molecules (ssRNA). Aptamers may be used to bind both nucleic acid and non-nucleic acid targets. Accordingly aptamers may be generated that recognise and so reduce the activity or expression of IFNα. Suitable aptamers may be selected from random sequence pools, from which specific aptamers may be identified which bind to the selected target molecules with high affinity. Methods for the production and selection of aptamers having desired specificity are well known to those skilled in the art, and include the SELEX (systematic evolution of ligands by exponential enrichment) process. Briefly, large libraries of oligonucleotides are produced, allowing the isolation of large amounts of functional nucleic acids by an iterative process of in vitro selection and subsequent amplification through polymerase chain reaction.

Antisense molecules represent another preferred agent for use according to the third or fourth aspects of the invention. Antisense molecules are typically single-stranded nucleic acids, which can specifically bind to a complementary nucleic acid sequence produced by a gene and inactivate it, effectively turning that gene “off”. The molecule is termed “antisense” as it is complementary to the gene's mRNA, which is called the “sense” sequence, as appreciated by the skilled person. Antisense molecules are typically are 15 to 35 bases in length of DNA, RNA or a chemical analogue. Antisense nucleic acids have been used experimentally to bind to mRNA and prevent the expression of specific genes. This has lead to the development of “antisense therapies” as drugs for the treatment of cancer, diabetes and inflammatory diseases. Antisense drugs have recently been approved by the US FDA for human therapeutic use. Accordingly, by designing an antisense molecule to polynucleotide sequence encoding IFN polypeptide it would be possible to reduce the expression of IFNα polypeptide in a cell and thereby reduce in IFNα activity and reduce the preactiviation seen in HCV infection. A polynucleotide sequence encoding an IFNα polypeptide is provided in FIG. 8.

Small interfering RNA (siRNA), sometimes known as short interfering RNA or silencing RNA, represent further preferred agents for use according to the third or fourth aspects of the invention. As set out above, the inventors realised that preactivation of the IFN system is associated with a resistance to antiviral therapy. It is therefore apparent that siRNA molecules that can reduce IFNα expression have great utility in the preparation of medicaments for the treatment of HCV infection. siRNA are a class of 20-25 nucleotide-long RNA molecules are involved in the RNA interference pathway (RNAi), by which the siRNA can lead to a reduction in expression of a specific gene, or specifically interfere with the translation of such mRNA thereby inhibiting expression of protein encoded by the mRNA. siRNAs have a well defined structure: a short (usually 21-nt) double-strand of RNA (dsRNA) with 2-nt 3′ overhangs on either end. Each strand has a 5′ phosphate group and a 3′ hydroxyl (—OH) group. In vivo this structure is the result of processing by Dicer, an enzyme that converts either long dsRNAs or hairpin RNAs into siRNAs. siRNAs can also be exogenously (artificially) introduced into cells by various transfection methods to bring about the specific knockdown of a gene of interest. Essentially any gene of which the sequence is known can thus be targeted based on sequence complementarity with an appropriately tailored siRNA. Given the ability to knockdown essentially any gene of interest, RNAi via siRNAs has generated a great deal of interest in both basic and applied biology. There is an increasing number of large-scale RNAi screens that are designed to identify the important genes in various biological pathways. As disease processes also depend on the activity of multiple genes, it is expected that in some situations turning off the activity of a gene with a siRNA could produce a therapeutic benefit. Hence their discovery has led to a surge in interest in harnessing RNAi for biomedical research and drug development. Recent phase I results of therapeutic RNAi trials demonstrate that siRNAs are well tolerated and have suitable pharmacokinetic properties. siRNAs and related RNAi induction methods therefore stand to become an important new class of drugs in the foreseeable future. siRNA molecules designed to nucleic acid encoding IFNα polypeptide can be used to reduce the expression of IFNα and therefore result in a reduction in the preactivation of the IFN system. Hence an embodiment of this aspect of the invention is wherein the agent is a siRNA molecule having complementary sequence to IFNα polynucleotide.

A polynucleotide sequence encoding an IFNα polypeptide is provided in FIG. 8.

Using such information it is straightforward and well within the capability of the skilled person to design siRNA molecules having complementary sequence to IFNα polynucleotide. For example, a simple internet search yields many websites that can be used to design siRNA molecules.

By “siRNA molecule” we include a double stranded 20 to 25 nucleotide-long RNA molecule, as well as each of the two single RNA strands that make up a siRNA molecule.

It is most preferred that the siRNA is used in the form of hairpin RNA (shRNA). Such shRNA may comprise two complementary siRNA molecules that are linked by a spacer sequence (e.g. of about 9 nucleotides). The complementary siRNA molecules may fold such that they bind together.

A ribozyme capable of cleaving RNA or DNA encoding IFNα polypeptide represent another preferred agent of the third or fourth aspect of the invention.

It is preferred that the agent of the third or fourth aspect of the invention is able to reduce the activation of the IFN system in a subject to be treated but not to reduce the activity of subsequent antiviral therapy supplied to the subject.

For example, where the agent of the third or fourth aspect of the invention is an antibody or fragment thereof, then it is preferred that the agent can bind to and reduce the activity of endogenous IFNα polypeptide but not exogenously supplied IFNα polypeptide. It is possible to derive such antibodies using methods routine in the art, and the information provided previously in this aspect of the invention.

It will be appreciated that the amount of an agent needed according to the invention is determined by biological activity and bioavailability which in turn depends on the mode of administration and the physicochemical properties of the agent. The frequency of administration will also be influenced by the abovementioned factors and particularly the half-life of the agent within the target tissue or subject being treated.

Known procedures, such as those conventionally employed by the pharmaceutical industry (e.g. in vivo experimentation, clinical trials etc), may be used to establish specific formulations of the agents and precise therapeutic regimes (such as daily doses and the frequency of administration).

Generally, a daily dose of between 0.01 μg/kg of body weight and 0.1 g/kg of body weight of an agent may be used in a treatment regimen for treating HCV infection; more preferably the daily dose is between 0.01 mg/kg of body weight and 100 mg/kg of body weight.

By way of example a suitable dose of an antibody according to the invention is 10 μg/kg of body weight—100 mg/kg of body weight, more preferably about 01 mg/kg of body weight-10 mg/kg of body weight and most preferably about 6 mg/kg of body weight.

Daily doses may be given as a single administration (e.g. a single daily injection or a single dose from an inhaler). Alternatively the agent (e.g. an antibody or aptamer) may require administration twice or more times during a day.

Medicaments according to the invention should comprise a therapeutically effective amount of the agent and a pharmaceutically acceptable vehicle.

A “therapeutically effective amount” is any amount of an agent according to the invention which, when administered to a subject inhibits or prevents cancer growth or metastasis.

A “subject” may be a vertebrate, mammal, domestic animal or human being. It is preferred that the subject to be treated is human. When this is the case the agents may be designed such that they are most suited for human therapy (e.g. humanisation of antibodies as discussed above). However it will also be appreciated that the agents may also be used to treat other animals of veterinary interest (e.g. horses, dogs or cats).

A “pharmaceutically acceptable vehicle” as referred to herein is any physiological vehicle known to those skilled in the art as useful in formulating pharmaceutical compositions.

In one embodiment, the medicament may comprise about 0.01 μg and 0.5 g of the agent. More preferably, the amount of the agent in the composition is between 0.01 mg and 200 mg, and more preferably, between approximately 0.1 mg and 100 mg, and even more preferably, between about 1 mg and 10 mg. Most preferably, the composition comprises between approximately 2 mg and 5 mg of the agent.

Preferably, the medicament comprises approximately 0.1% (w/w) to 90% (w/w) of the agent, and more preferably, 1% (w/w) to 10% (w/w). The rest of the composition may comprise the vehicle.

Nucleic acid agents can be delivered to a subject by incorporation within liposomes, Alternatively the “naked” DNA molecules may be inserted into a subject's cells by a suitable means e.g. direct endocytotic uptake. Nucleic acid molecules may be transferred to the cells of a subject to be treated by transfection, infection, microinjection, cell fusion, protoplast fusion or ballistic bombardment. For example, transfer may be by ballistic transfection with coated gold particles, liposomes containing the DNA molecules, viral vectors (e.g. adenovirus) and means of providing direct DNA uptake (e.g. endocytosis) by application of the DNA molecules directly to the target tissue topically or by injection.

The antibodies, or functional derivatives thereof, may be used in a number of ways. For instance, systemic administration may be required in which case the antibodies or derivatives thereof may be contained within a composition which may, for example, be ingested orally in the form of a tablet, capsule or liquid. It is preferred that the antibodies, or derivatives thereof, are administered by injection into the blood stream. Injections may be intravenous (bolus or infusion) or subcutaneous (bolus or infusion). Alternatively the antibodies may be injected directly to the liver.

Nucleic acid or polypeptide therapeutic entities may be combined in pharmaceutical compositions having a number of different forms depending, in particular on the manner in which the composition is to be used. Thus, for example, the composition may be in the form of a powder, tablet, capsule, liquid, ointment, cream, gel, hydrogel, aerosol, spray, micelle, transdermal patch, liposome or any other suitable form that may be administered to a person or animal. It will be appreciated that the vehicle of the composition of the invention should be one which is well tolerated by the subject to whom it is given, and preferably enables delivery of the therapeutic to the target cell, tissue, or organ.

In a preferred embodiment, the pharmaceutical vehicle is a liquid and the pharmaceutical composition is in the form of a solution. In another embodiment, the pharmaceutical vehicle is a gel and the composition is in the form of a cream or the like.

Compositions comprising such therapeutic entities may be used in a number of ways. For instance, systemic administration may be required in which case the entities may be contained within a composition that may, for example, be ingested orally in the form of a tablet, capsule or liquid. Alternatively, the composition may be administered by injection into the blood stream. Injections may be intravenous (bolus or infusion) or subcutaneous (bolus or infusion). The entities may be administered by inhalation (e.g. intranasally).

Therapeutic entities may also be incorporated within a slow or delayed release device. Such devices may, for example, be inserted on or under the skin, and the compound may be released over weeks or even months. Such devices may be particularly advantageous when long term treatment with an entity is required and which would normally require frequent administration (e.g. at least daily injection).

The agents of the first aspect of the invention are particularly useful for pretreating patients about to undergo treatment with antiviral therapy with IFN (e.g. pegIFN) and an antiviral agent such as ribavirin. It is therefore preferred that the agent is administered to a virally infected individual before therapy with IFN and ribavirin is initiated.

The length of time between the pre-treatment with the agents defined in relation to the third and fourth aspect of the invention and the antiviral therapy can depend on the agents used. For example, where the agent is able to reduce the activation of the IFN system in a subject to be treated but not to reduce the activity of subsequent antiviral therapy supplied to the subject, then the length of time can be very short. For example, the subject could be treated concurrently, or even with a combined treatment regime.

If the agent is not distinguishing, then the length of time can depend on the nature of the agent. For example, it is known that exogenously supplied antibody takes around 4 to 6 weeks in order to be cleared from the human body. Therefore, where the agent is an antibody to the IFNα polypeptide or receptor, or other such member of the IFN system, then preferably the subsequent antiviral therapy is supplied to the patient 4 to 6 weeks later, preferably at least 6 weeks.

The various elements required for a technician to perform the method of the first aspect of the invention may be incorporated in to a kit.

Thus, according to a fifth aspect of the invention there is provided a kit for determining the likelihood that a subject having a viral infection of the liver will be responsive to antiviral therapy that includes stimulation of Interferon (IFN) activity, comprising:

(i) means for analysing in a sample from a subject the expression of at least one gene from each of the groups of genes listed above and shown in Table 2; and, optionally,
(ii) means for comparing expression of the genes in the sample to expression of the same genes in a control sample.

By “means for analysing in a sample from a subject the expression of at least one gene from each of the groups of genes listed above and shown in Table 2” we include the specific binding molecules given in the first aspect of the invention that can target molecules representative of gene expression in the sample. Preferably the specific binding molecule is an oligonucleotide probe, antibody, aptamers, or binding proteins or small molecules mentioned above.

By “means for comparing expression of the genes in the sample to expression of the same genes in a control sample” we include the control samples mentioned above in the first aspect of the invention. We also include the control reference data mentioned therein.

The kit of the fifth aspect of the invention may also comprise:

(iii) relevant buffers and regents for analysing the expression of said genes.

The buffers and regents provided with the kit may be in liquid form and preferably provided as pre-measured aliquots. Alternatively, the buffers and regents may be in concentrated (or even powder form) for dilution.

The various elements required for a technician to perform the method of the second aspect of the invention may be incorporated in to a kit.

Thus, according to a sixth aspect of the invention there is provided a kit for determining the likelihood that a subject having a viral infection of the liver will be responsive to antiviral therapy that includes stimulation of Interferon (IFN) activity, comprising means for examining a sample from the subject to identify the subcellular location of STAT1.

By “means for examining a sample from the subject to identify the subcellular location of STAT1” we include the specific binding molecules given in the second aspect of the invention that can identify the subcellular location of STAT1. Preferably said specific binding molecule is an anti-STAT antibody; preferably an anti-phospho-STAT1 antibody.

The kit of the sixth aspect of the invention may also comprise:

(iii) relevant buffers and regents for identifying the subcellular location of STAT1.

The buffers and reagents provided with the kit may be in liquid form and preferably provided as pre-measured aliquots. Alternatively, the buffers and regents may be in concentrated (or even powder form) for dilution.

All of the features described herein (including any accompanying claims, abstract and drawings), and/or all of the steps of any method or process so disclosed, may be combined with any of the above aspects in any combination, except combinations where at least some of such features and/or steps are mutually exclusive.

The invention will now be further described with reference to the following Example and figures in which:

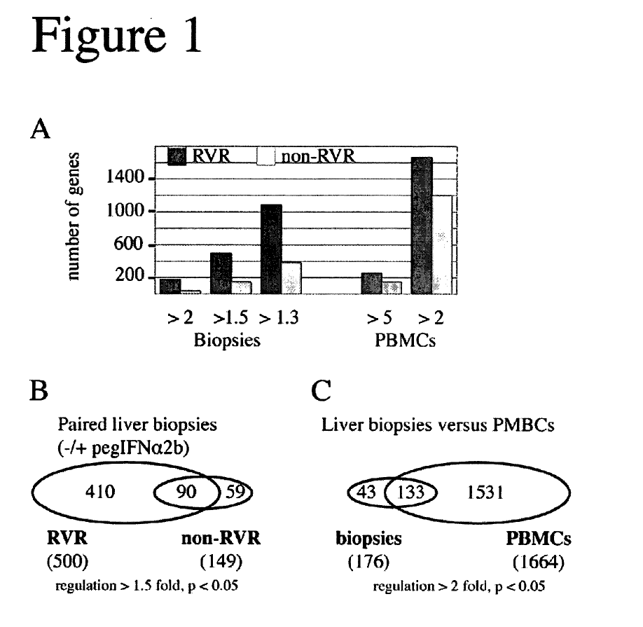

FIG. 1. PegIFN-α2b induced regulation of gene expression in liver and PBMCs.

(A) Rapid responders up- or down-regulate significantly more genes in response to pegIFN-α2b than non-RVR patients. Shown are numbers of genes significantly changed in >60% of patients (p<0.05)>2-, >1.5- or >1.3-fold in liver and >5- and >2fold in PBMCs.
(B) Venn diagram of genes significantly (p<0.05) up- or down-regulated >1.5-fold in response to pegIFN-α in at least 60% of patients in the RVR versus the non-RVR group.
(C) Venn diagram of genes significantly (p<0.05) up- or down-regulated >2-fold in response to pegIFN-α in at least 60% of patients in liver versus PBMCs of RVR patients.

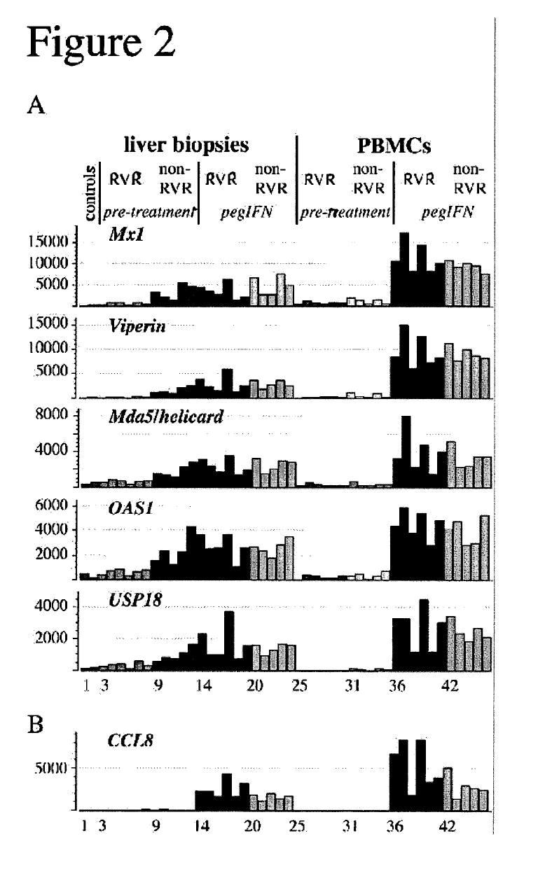

FIG. 2. PegIFN-α2b induced gene regulation in HCV-infected patients show s major differences between livers of RVR and non-RVR patients and between liver and PBMCs.

(A) Five ISGs (Mx1, viperin, Mda5/helicard, OAS1, USP18) were chosen from the list of genes significantly regulated >1.5-fold between B-1 and B-2 in RVR patients. In liver of non-RVR patients, expression of these genes is already high before treatment (lanes 9-13), and does not further increase after pegIFNα (lanes 20-24). In RVR patients, pre-treatment expression (lanes 3-8) is similar to controls (lanes 1-2), and pegIFNα induces a strong upregulation (lanes 3-8 versus 14-19). No pre-activation is found in PBMCs (lanes 25-46). The y-axes display raw expression values.
(B) An example of a gene (CCL8) upregulated in liver in response to pegIFN-α2b in both RVR and non-RVR patients.

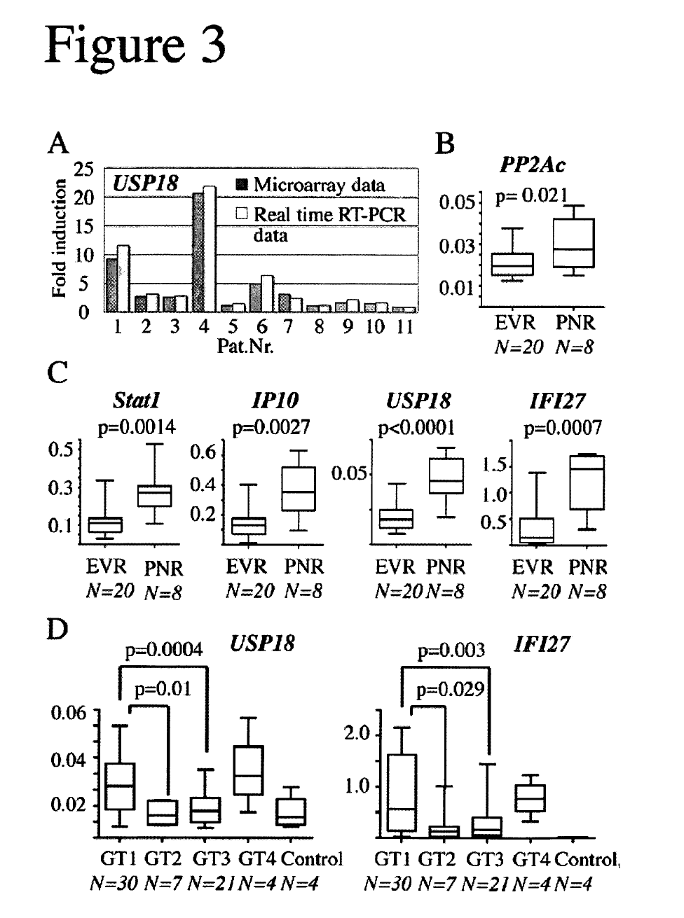

FIG. 3. RT-qPCR analysis of selected ISGs and of the catalytic subunit of PP2A.

(A) RT-qPCR analysis of the USP18 mRNA corroborates the array data. Depicted is the fold induction of USP18 mRNA between B-1 and B-2 in individual patients.
(B) PP2Ac expression is significantly lower in EVR than PNR patients.
(C) The expression level of selected ISGs in pre-treatment biopsies is lower in EVR than in PNR patients.
(D) Expression levels of USP18 and IFI27 in pre-treatment biopsies are significantly higher in patients infected with genotype 1 compared to genotypes 2 or 3. In panels B, C and D, the y axis shows expression relative to that of GAPDH. Statistical significance was tested with the unpaired t-test for USP18 and with the Mann-Whitney test for all other genes. N=number of patients in each group.

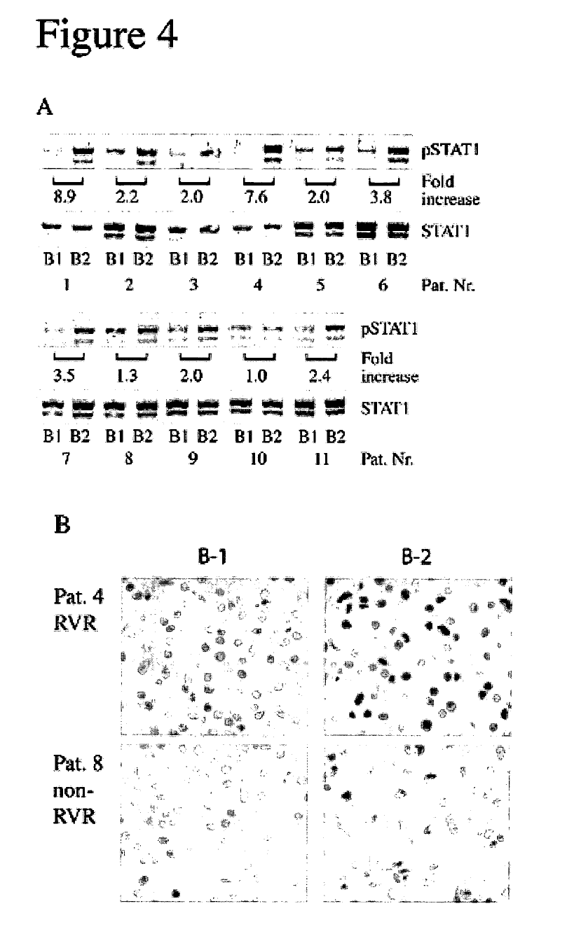

FIG. 4. Analysis of Jak-STAT signaling in liver biopsies.

(A) STAT1 phosphorylation in extracts of liver biopsies collected before (B-1) and after (B-2) pegIFN-α2b treatment. Extracts were analyzed by Western blot using antibodies specific for PY(701)-STAT1. Most patients showed increase in phosphorylated STAT1 in the liver in response to pegIFN-α2b. The signals were quantified using the Odyssey Imaging Software to calculate the integrated intensity (kilo counts×mm²). The values represent the fold increase of phosphorylation in B-2 samples. Blots were stripped and reprobed for total STAT1 used as a loading control for each pair of samples.
(B) Immunohistochemical staining of phospho-STAT1 in liver biopsies reveals a weak nuclear staining in pre-treatment biopsies of non-RVR patients.

Shown are representative examples of B-1 and B-2 of RVR and non-RVR patients. No nuclear staining is evident in pre-treatment biopsies of RVR patients (Pat. 4). The light blue color of nuclei originates from the counterstaining with haematoxilin. 4 h after pegIFNα, most hepatocytes of RVR patients show a strong nuclear staining. In non-RVR patients, a weak nuclear staining is already present in pre-treatment biopsies, and pegIFNα induces little changes in hepatocytes. The visible increased nuclear staining is confined to Kupffer cells.

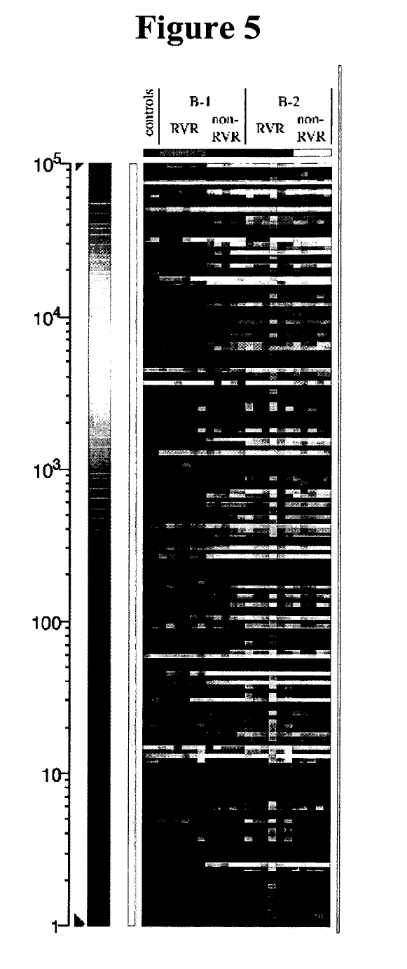

FIG. 5.

The predominant pattern of gene expression in all patient biopsy samples is shown as a heat map. The map was generated using a list of 176 genes that are altered >2 fold in at least 60% of RVRs with a p value of <0.05. The colour-coding of the raw expression values is shown on the left. Many genes have a low expression level in the control patients and the pre-treatment biopsies of the RVR patients (B-1), and comparable high expression levels in B-1 of non-RVR patients and in all B-2 samples.

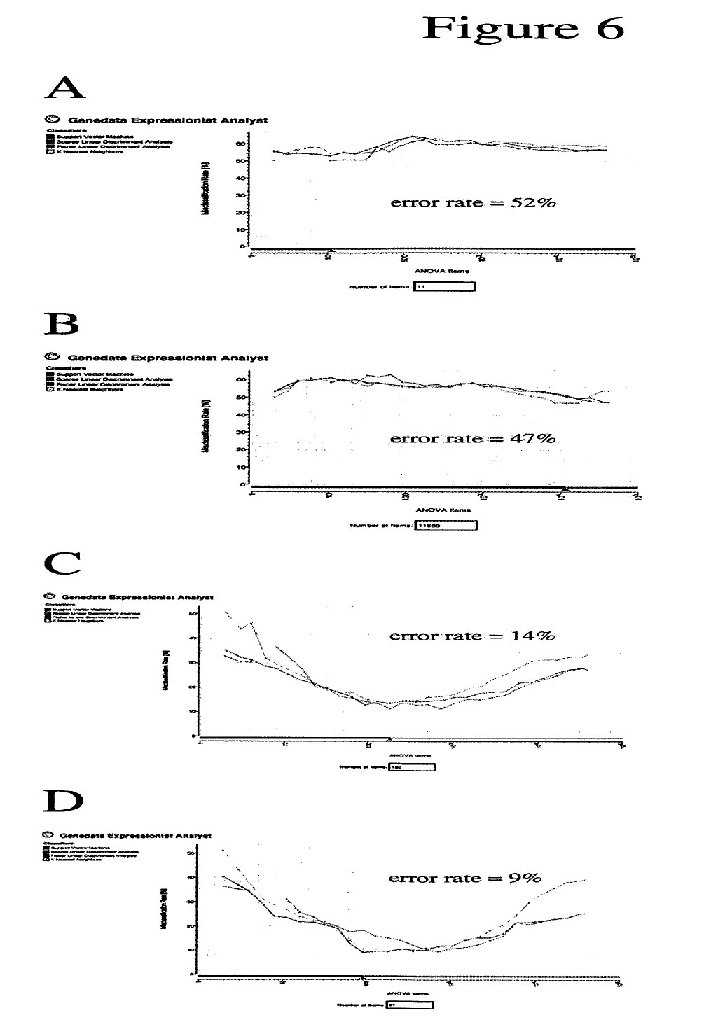

FIG. 6.

Supervised classifier prediction in liver biopsy samples and PBMCs with response to treatment at week 4 as grouping criterium.

(A and B) Supervised classifier prediction of PBMC-1 and PBMC-2 samples did not generate a useful list of predictive genes with any of the 4 statistical tests used (Support Vector Machine, Sparse Linear Discriminant Analysis, Fisher Linear Discriminant Analysis, K Neirest Neighbors). The misclassification rates were 52% for PBMC-1 and 47% for PBMC-2.
(C) Supervised classifier prediction using the B-2 biopsies of the two response groups revealed a list of 173 genes (180 transcripts) as best predictors of treatment outcome with a misclassification rate of 14%.
(D) Supervised classifier prediction using the B-1 biopsies of the two response groups resulted in a list of 83 genes (91 transcripts) as best predictors of treatment outcome with a misclassification rate of 9%.

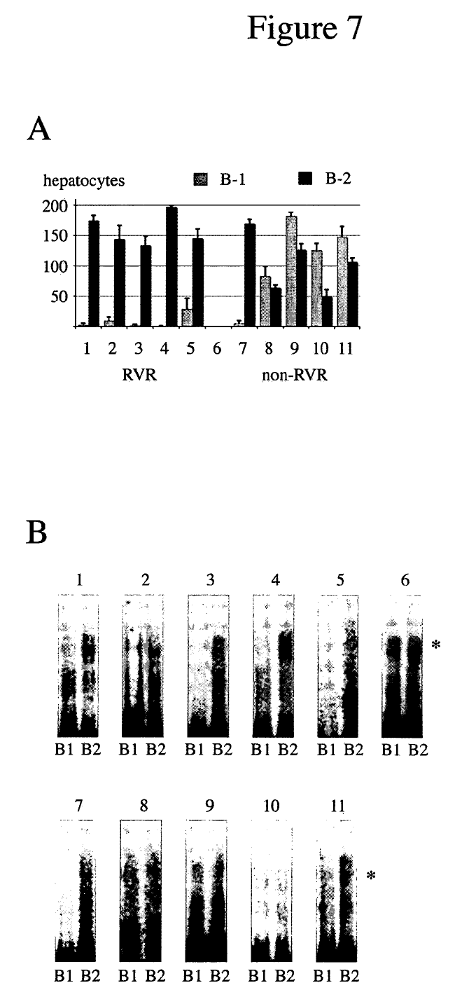

FIG. 7

(A) Semiquantitative assessment of immunohistochemical staining of phospho-STAT1 in liver biopsies. Nuclear staining of hepatocytes was quantified by repeated counting (5 times) in 200 hepatocytes in B-1 (blue) and B-2 (red) samples of the indicated patients (patient numbers correspond to the numbers in table 1). In four out of five non-RVR patients, a considerable proportion of hepatocytes had a weak but clear nuclear staining already in the pre-treatment biopsies. All the RVR patients had no phospho-STAT1 signals in the nuclei before treatment, but showed a strong induction after pegIFNα.
(B) The induction of STAT-DNA binding in response to pegIFNα2b is impaired in most of the non-RVR patients. Nuclear extracts from B-1 and B-2 samples were analyzed with EMSAs using the radiolabeled SIE-m67 oligonucleotide probe. The asterisk (*) depicts the signal of the activated STAT1 dimers that have bound the oligonucleotide sequence. The numbers above the gel shift panels represent the patient numbers. The upper panel shows the 6 patients with a rapid response at week 4 (numbers 1-6). The lower panel shows the 5 patients without a virological response at week 4 (numbers 7-11).

FIG. 8: Amino acid and nucleotide sequences of human Interferon α.

FIG. 9: Amino acid and nucleotide sequence of human Interferon Receptor 1.

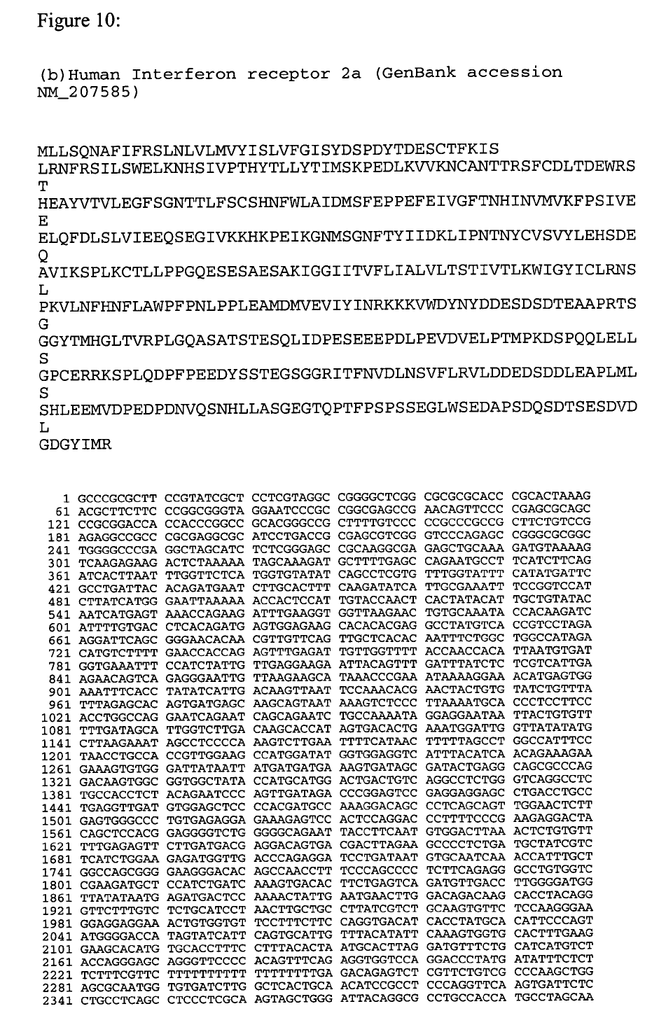

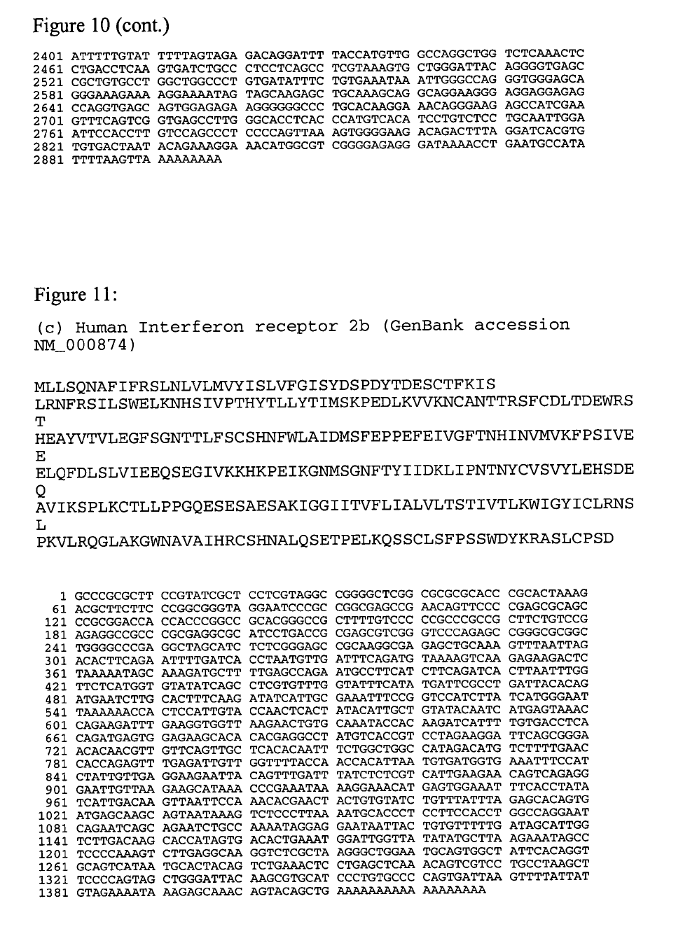

FIG. 10: Amino acid and nucleotide sequence of human Interferon Receptor 2.

FIG. 11: Amino acid and nucleotide sequence of human Interferon Receptor 2b.

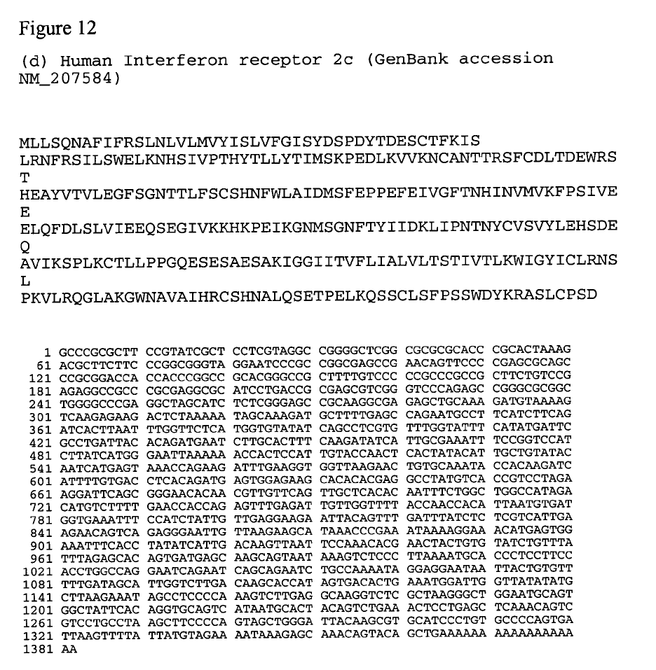

FIG. 12: Amino acid and nucleotide sequence of human Interferon Receptor 2c.

### EXAMPLE 1

### 1.1 Methods

### 1.1.1 Subject Samples and Treatment

Paired human liver biopsy samples from 11 chronically infected HCV subjects were obtained. From January 2006 to April 2007, all subjects with chronic hepatitis C referred to the outsubject liver clinic of the University Hospital Basel were asked for their permission to use part of their diagnostic liver biopsy for research purposes. Liver biopsies were obtained by ultrasound-guided technique using a coaxial needle.

After removal of two 20- to 25-mm long biopsy specimens for routine histopathological workup for grading and staging of the liver disease according to the Metavir scoring system, the remaining 5- to 20-mm long biopsy cylinders were labeled as B1 (for biopsy 1) and stored as pretreatment samples of future study participants. Pegylated-IFNα2b (Essex Chemie AG, Switzerland) was prescribed to all subjects participating in this study. Second biopsy (B2) was performed 4 hours following the first pegIFNα2b injection. The first dose of ribavirin was given after this second biopsy to avoid further confounding factors. The protocol was approved by the Ethics Committee of the University Hospitals in Basel. Written informed consent was obtained from all subjects.

In addition, blood for peripheral blood mononuclear cell (PBMC) isolation was collected before treatment and 4 hours after the first pegIFNα2b injection.

The HCV subjects underwent a standard combination treatment with pegIFNα2b (1.5 μg/kg body weight) and ribavirin (weight based dosing: <65 kg: 800 mg/d; 65-85 kg: 1 g/d; <85 kg: 1.2 g/d). HCV-RNA was quantified before treatment initiation, at week 4 and week 12 of the treatment (Table 1). Treatment duration is 24 weeks for subjects with genotypes ⅔ and 48 weeks for genotype 1. From the 11 subjects included in the study, 2 subjects (Nr. 10 and 11) had a primary non-response and treatment was stopped at week 12. From the remaining 9 subjects, 2 (Nr. 1, 2) have accomplished the therapy with an end of treatment response.

As non-HCV controls, two subjects that underwent ultrasound-guided liver biopsies of focal lesions (metastasis of carcinomas) gave informed consent for a biopsy from the normal liver tissue outside the focal lesion. Again, a part of the biopsy was used for routine histopathological diagnosis, and the remaining tissue for the extraction of RNA, as described later. Both control samples showed confirmed absence of liver disease in the routine histopatholog ical workup.

### 1.1.2 Measurement of IFN Alpha Serum Concentrations

Pretreatment hIFNα serum levels and the serum concentration of pegIFNα2b 4 h after the first injection were measured using the human interferon alpha ELISA kit from PBL Biomedical Laboratories according to manufacturer's instructions. This ELISA kit has previously been shown to recognize both unpeg ylated and peg ylated human IFNα³⁴.

1.1.3 Preparation of Extracts from Human Liver Biopsies

Liver biopsy samples were used for the preparation of whole cell, cytoplasmic and nuclear extracts. For whole cell extracts, samples were dounce homogenized in 100 μl of lysis buffer containing 100 mmol/l NaCl, 50 mmol/l Tris pH 7.5, 1 mmol/l EDTA, 0.1% Triton X-100, 10 mmol/l NaF, 1 mmol/l phenylmethyl sulfonyl fluoride, and 1 mmol/l vanadate. Lysates were centrifuged at 14,000 rpm at 4° C. for 5 minutes. Protein concentration was determined by Lowry (BioRad Protein Assay).

For nuclear and cytoplasmic extracts, livers were lysed in a low-salt buffer containing 200 mmol/l Hepes pH 7.6, 10 mmol/l KCl, 1 mmol/l EDTA, 1 mmol/l EGTA, 0.2% NP-40, 10% glycerol, and 0.1 mmol/l vanadate. After centrifugation at 15,000 rpm for 5 minutes, the pellet was resuspended in high-salt buffer (low-salt buffer supplemented with 420 mmol/L NaCl). After centrifugation, aliquots of nuclear extracts were made for electrophoretic mobility shift assays (EMSAs).

1.1.4 Western blots and Electrophoretic Mobility Shift Assays

10 μg of total protein from human liver lysates was loaded for sodium dodecyl sulphate polyacrylamide gel electrophoresis and transferred onto a nitrocellulose membrane (Schleicher & Schuell, Bottmingen, Switzerland). The membranes were blocked in 3% BSA/milk (1:1)-0.1% Triton X-100 for 1 hour, washed with Tris-buffered saline Tween-20 (TBST), and incubated with the primary antibody overnight at 4° C.

Proteins were detected with primary antibodies specific to phosphorylated STAT1 (PY(701)-STAT1; Cell Signaling, Bioconcept, Allschwil, Switzerland) and STAT1 (carboxy-terminus; Transduction Laboratories, BD Biosciences, Pharmingen). After 3 washes with TBST, the membranes were incubated with infrared fluorescent secondary goat anti-mouse (IRDye 680) or anti-rabbit (IRDye 800) antibodies (both from LI-COR Biosciences) for 1 hour at room temperature. Blots were analyzed by the Odyssey Infrared Imaging System from LI-COR. The infrared image was obtained in a single scan and the signal was quantified using the integrated intensity.

For loading controls, the membranes were stripped and incubated with anti-β-Actin antibody (Sigma).

EMSAs were performed using 2 μg of nuclear extracts and ³²P-radiolabeled DNA-oligonucleotide serum inducible element (SIE)-m67 corresponding to STAT response element sequences²⁵.

### 1.1.5 Immunohistochemistry

Standard indirect immunoperoxidase procedures were used for immunohistochemistry (ABC-Elite, Vectra Laboratories). 4-mm-thick sections were cut from paraffin blocks, rehydrated, pretreated (20′ in ER2 solution) incubated with a monoclonal rabbit antibody against phospho-STAT1 (dilution 1:200, #9167 Cell Signaling) and counterstained with haematoxilin. The whole staining procedure (dehydration, pre-treatment, incubation, counterstaining and mounting) was performed with an automated stainer (Bond®, Vision BioSystems Europe, Newcastle-upon-Tyne, UK). For quantification of nuclear phospho-STAT1 staining, 5 times 200 hepatocytes were counted for each B-1 and B-2 sample of each patient. In supplementary FIG. 3, the mean values with the standard deviations are shown.

### 1.1.6 RNA Isolation and Microarray Analysis

Total RNA was extracted from liver and PBMC samples using the RNeasy Mini Kit (Qiagen) according to manufacturer's instructions. RNA was aliquoted and stored at −80° C. Gene expression was assessed in liver and PBMCs by microarray analysis using Affymetrix Human Genome U133 Plus 2.0 arrays representing over 56,000 transcripts and variants with 11 perfect-match/mis-match probe pairs per transcript. The microarray hybridizations were performed at the functional genomics facility of the Friedrich Miescher Institute for Biomedical Research in Basel. Total RNA (1-2 μg) from each sample was reverse transcribed and biotinylated using the Affymetrix 1-cycle amplification kit as per manufacturer's instructions. Biotinylated cRNA (20 μg) was fragmented by heating with magnesium (as per Affymetrix's instructions) and 15 μg of fragmented cRNA was hybridized to Human U133 Plus 2.0 GeneChips according to the manufacturer's instructions. Quality control and background normalization was performed using Refiner 4.1 from Genedata AG (Basel, Switzerland). Expression value estimates were obtained using the GC-RMA implementation in Refiner 4.1. LOWESS-normalization and median scaling of the genes called present (detection P-value <0.04) to a value of 500 was performed in Genedata's Analyst 4.1 package. The LOWESS-normalized data are referred to as ‘raw’ expression values in this paper. We also performed a point-wise division on the genes by dividing each gene by its median in order to centre its expression level at 1.0. This scaled data shows only the magnitude and direction of change but not of absolute expression level. Scaled data were used for clustering analyses. Unless noted, all other analyses were performed using the raw data.

Data analysis was performed using Expressionist® Analyst 4.1 from Genedata AG. Genes were required to pass a t-test with a P<0.05 and have a median fold change of 1.3, 1.5, 2 and 5 or greater between the paired patient samples in at least 60% of patients within each group. For the supervised classifier prediction of liver biopsy samples and PBMCs using the response at week 4 as a grouping criterion 4 statistical tests were used (Support Vector Machine, Sparse Linear Discriminant Analysis, Fisher Linear Discriminant Analysis, K Nearest Neighbors). The misclassification rates could be determined for every test used and the one with the lowest rate was selected.

### 1.1.7 RNA Isolation, Reverse Transcription and SYBR-PCR

The array data were validated by quantitative real-time RT-PCR analysis of several IFN regulated genes including STAT1, IP10, USP18, IFI27 SOCS1 and SOCS3.

Total RNA was extracted from liver using the RNeasy Mini Kit (Qiagen) according to manufacturer's instructions. The RNA was reverse transcribed by Moloney murine leukemia virus reverse transcriptase (Promega Biosciences, Inc., Wallisellen, Switzerland) in the presence of random hexamers (Promega) and deoxynucleoside triphosphate. The reaction mixture was incubated for 5 min at 70° C. and then for 1 h at 37° C. The reaction was stopped by heating at 95° C. for 5 min. SYBR-PCR was performed based on SYBR green fluorescence (SYBR green PCR master mix; Applied Biosystems, Foster City, Calif.). Primers for GAPDH (glyceraldehyde-3-phosphate dehydrogenase), STAT1, inducible protein 10 (IP10), SOCS1, SOCS3, USP18, IF127 and PP2Ac were designed across exon-intron junctions. The primer sequences are shown in Table 4. The difference in the cycle threshold (ΔC_{T}) value was derived by subtracting the C_{T} value for GAPDH, which served as an internal control, from the C_{T} value for STAT1 or other transcripts of interest. All reactions were run in duplicate by using an ABI 7000 sequence detection system (Applied Biosystems). mRNA expression levels of the transcripts were calculated relative to GAPDH from the ΔC_{T} values using the formula 2-ΔCT. The change of expression in paired liver biopsy samples was calculated as a fold change according to the formula 2̂(ΔC_{T}B-1−ΔC_{T}B-2).

Box plot diagrams, unpaired t-tests and Mann Whitney tests were performed using GraphPad Prism version 4.00 for Macintosh, GraphPad Software, San Diego Calif. USA, www.graphpad.com.

### 1.2 Results

### Patients and Response to Treatment

11 patients included in this study, 4 women and 7 men, were treated with a weight-adjusted combination of subcutaneously injected pegIFNα2b once weekly and oral ribavirin twice daily. All of them had two liver biopsies, the pre-treatment biopsy (B-1) and the second biopsy (B-2) obtained 4 h after the first injection of pegIFNα2b. We have chosen to analyze gene expression 4 h after pegIFNα2b injection since kinetics of the induction of ISGs by pegIFNα in liver of chimpanzees was maximal at this time and was followed by a rapid down-regulation of many genes (22). We realize that we probably missed the up-regulation of some late induced ISGs, but because of the rapid down-regulation, we would have missed more ISGs when using later time-points.

Five of the patients were infected with HCV genotype 1, and 3 patients each with genotype 2 and 3 (Table 1). 6 patients had negative serum HCV RNA after 4 weeks of treatment and were classified as RVRs, whereas 5 patients were still positive for HCV RNA at week 4 (non-RVRs). All 6 patients with an RVR were still HCV RNA negative at week 12 (EVRs). 2 of the 5 non-RVR patients showed >2 log₁₀ drop of the viral load after 12 weeks of therapy, and were classified as EVR. 3 patients were non-responders at week 12 (primary non-responders, PNR) (Table 1).

Serum IFNα concentrations were below the limit of detection in all patients before treatment, and, in accordance with previously published pharmacokinetic data (24), between 34 and 360 μg/ml in samples obtained at 4 h after the pegIFNα2b injection (data not shown). There was no significant correlation between the virological response at week 4 and the serum IFNα concentration at 4 h post-injection. Furthermore, despite the differences in the serum IFNα levels, all patients showed similar ISG induction in PMBCs (see below).

### IFN-Induced Regulation of Target Genes

Gene expression was analyzed with Affymetrix U133plus2.0 arrays in B-1 and B-2 samples, and also in PBMCs isolated from blood obtained before (PBMC-1) and 4 h after the first pegIFNα2b injection (PBMC-2). For each patient, the genes that were up- or down-regulated more than 1.3-, 1.5-, 2- or 5-fold in post-treatment samples were identified and saved in gene lists. These lists were used to identify all genes that were significantly (p<0.05) up- or down-regulated in at least 60% of the patients in the RVR and non-RVR groups. In this analysis, RVR patients were found to up- or down-regulate a larger number of genes than patients without a RVR (FIG. 1A). For example, in the RVR group, 500 genes were significantly changed >1.5-fold, but only 149 in the non-RVR samples. Overall, 3-5 times more genes were induced in the liver within the first 4 h after pegIFNα2a injections in RVR versus non-RVR patients. There was also a difference in the PBMC samples, but only between 1.4- and 1.8-fold (FIG. 1A). There was an overlap in the significantly regulated genes found in RVR samples and non-RVR samples. For example, 90 of the 149 genes that changed more >1.5-fold in non-RVR samples were also present among 500 genes changed in the RVR group (FIG. 1B).

Not surprisingly, many of the regulated genes represent known ISGs. However, contrary to our expectations, expression levels of these ISGs were not higher in post pegIFNα2b treatment biopsies from RVR patients as compared to non-RVRs. Rather, non-RVR patient samples had a higher level of ISG expression already in B-1, and the fold change in the B-2 samples was therefore only minor. This is illustrated in FIG. 2A at the example of five ISGs: Mx1, viperin, Mda5/helicard, oligoadenylate synthetase 1 (OAS1), and USP18. The genes show a very low expression in biopsies from individuals without hepatitis C and in B-1's of RVR patients. The 5 non-RVR patients had high expression of these genes before treatment, and pegIFNα2a administration not or only minimally increased their expression. There were very few exceptions to this rule (an example is shown in FIG. 2B). These genes had low expression in the pre-treatment biopsies, and pegIFNα2b induced them in all patients. Nevertheless, the predominant pattern of gene expression resembled this shown in FIG. 2A. A heat map of the expression of 176 genes significantly changed >2 fold between B-1 and B-2 in the RVR group has been done for all biopsy samples.

There was a considerable overlap of pegIFNα2b-regulated genes in liver and PBMCs (FIG. 1C). 133 of the 176 ISGs significantly regulated >2-fold in RVRs in the liver were also significantly changed in the PBMCs of these patients. Interestingly, in all patients pegIFNα2b regulated more genes in PBMCs than in liver. However, there was no significant difference in the upregulation of ISGs in PBMCs between RVRs and non-RVRs. No pre-activation of ISGs was found in PBMCs of non-RVRs, and pegIFNα2b treatment had the same effect on ISG regulation in RVR and non-RVR patients (for representative ISG examples, see FIG. 2). This indicates that chronic HCV infection has strong local effects on the IFN system in liver, but little effect in PBMCs.

A Subset of Genes that Predicts Response to Treatment

Supervised classifier analysis of array data allows the identification of a subset of genes that best predicts the outcome, in our case rapid response versus non-response at week 4. All liver biopsy and PBMC data sets were subjected to supervised classifier prediction using the response at 4 weeks of treatment as grouping criteria. For PBMC samples the analysis did not identify a subset of genes that could predict the treatment outcome. In contrast, a subset of 173 genes was identified in the liver B-2 samples that allowed to predict the response to treatment with an error rate of 14%. Even better prediction was possible with a subset of 83 genes in the pre-treatment biopsies B-1 where the error rate was 9%. In this set there were 22 genes up-regulated and 5 genes down-regulated by pegIFNα2b (Table 2). Therefore, 27 of the 83 (33%) best predictor genes represent IFN-regulated genes.

Contrary to the predominance of IFN-regulated genes in the best predictor set from pre-treatment biopsies, only few IFN-regulated genes were found in the set of best predictor genes derived from an analysis of the B-2 biopsies. Of the 173 different genes in this set, only 4 known IFN-regulated genes were up-regulated and 1 was down-regulated by pegIFNα2b (Table 3). These results support the findings shown in FIG. 2 that expression levels of the IFN-regulated genes in B-2 do not differ between RVR and non-RVR samples and therefore are not suited for the discrimination of responders from non-responders. Among the non-IFN-regulated genes present in the B1 and B2 liver biopsies lists discussed above are genes having functions in signal transduction, cell cycle regulation, apoptosis, and amino acid and lipid metabolism.

### RT-QPCR Analysis of ISG Expression in Liver Biopsies

Array analysis of the paired liver biopsies emphasized the importance of ISG expression in B-1 biopsies for the outcome of therapy. To confirm these data, we measured by real time quantitative PCR (RT-qPCR) the expression of selected ISGs (USP18, Stat1, IP10, IF127) in 11 patients with B1 and B2 biopsies, and in pre-treatment biopsies of 51 additional patients with CHC. In the 11 patients with the paired biopsies, the RT-qPCR values matched well the array expression, validating the quality of the array data (FIG. 3A, and data not shown). The expression of all four ISGs in pre-therapy biopsies was significantly different between the EVR and PNR groups (FIG. 3C), further supporting the conclusion that there is an inverse correlation between the pre-treatment expression of ISGs in liver and the response to IFNα therapy.

Pre-Treatment ISG Expression Levels Correlate with HCV Genotype

We also analyzed the expression of selected ISGs with regard to the HCV genotype (FIG. 3D). Interestingly, the investigated ISGs showed significantly higher expression in patients infected with the “difficult-to-treat” genotypes 1 and 4 than with genotypes 2 and 3, which can be successfully treated in over 80% of patients. Increased expression of ISGs in liver of patients infected with HCV genotypes 1 and 4 provides a plausible explanation for the poor response of these patients to IFN therapy.

Non-Responders have Higher Expression of PP2Ac

We have previously shown that the catalytic subunit of PP2A (PP2Ac) is over-expressed in liver of patients with CHC compared to controls, and that over-expression of PP2Ac inhibits IFNα signaling (14, 25). We therefore analyzed the PP2Ac mRNA levels in a group of patients with known treatment responses at week 12. Patients of the EVR group expressed significantly less PP2Ac mRNA than PNR patients (FIG. 3B).

### IFN-Induced Jak-STAT Signaling

The injected pegIFNα2b binds to IFN receptors and activates the Jak-STAT pathway. A central event in this activation is the phosphorylation of STAT1 on tyrosine 701 (26). We analyzed extracts from all B-1 and B-2 biopsies by Western blot using a phospho-specific STAT1 antibody (FIG. 4A). In most patients, STAT1 phosphorylation was induced in response to pegIFNα2b injection. The strongest induction was observed in two patients (1 and 4) later RVRs, while the weakest induction was found in patients 8 and 10 who did not clear the virus within the first 4 weeks. However, the remaining patients had similar 2- to 3.8-fold induction regardless of their virological response at week 4. Hence, STAT1 phosphorylation is not significantly impaired in non-RVR patients.

Phosphorylated STAT1 translocates into the nucleus and binds as a dimer to specific response elements of ISG promoters (26). Assessment of nuclear translocation by immunohistochemistry, using anti-phospho-STAT1 antibodies, should potentially allow to discriminate between STAT1 activation in hepatocytes and other cells present in the biopsy material. Analysis of paired biopsies of RVR patients revealed a minimal nuclear staining in B-1 samples and a strong staining in most hepatocyte nuclei in B-2 samples, following injection of pegIFNα (FIG. 4B). In contrast, all but one (number 7) non-RVR patients showed a remarkably different staining pattern. In the pre-treatment biopsies, a large proportion of hepatocytes already had an appreciable nuclear staining, which did not increase in B-2 samples. The visible increase in nuclear staining in B-2 samples of non-RVR patients originated from nuclear translocation of STAT1 in Kupffer cells (liver macrophages), and not hepatocytes (FIG. 4B). Activation of STAT1 in Kupffer cells, and possibly contaminating blood cells, may have contributed to the increased STAT1 phosphorylation observed in Western blotting (FIG. 4A).

The next step in the signaling pathway is the binding of nuclear phospho-STAT1 to promoter elements of ISGs. We therefore assessed the STAT1 DNA-binding in extracts of B-1 and B-2 biopsies by performing electrophoretic mobility shift assays (EMSAs). All rapid responders showed a marked increase in the STAT1 DNA binding in the B-2 samples. In contrast, most non-RVR patients showed a minimal or no increase of the gel shift signal upon pegIFNα application.

These data indicate that results of immunohistochemistry and EMSA assays correlate better with the therapy outcome than results of Western analysis for phospho-STAT1. Taken together, the data demonstrate substantial differences in the IFN-induced Jak-STAT signaling between RVR and non-RVR patients.

### 1.3. Discussion

To learn more about possible mechanisms underlying differential response of HCV-infected patients to IFN therapy, we investigated the IFN-induced signaling and ISG induction in paired liver biopsies collected from patients with CHC before and during therapy pegIFNα. Comparison of IFN signaling in two liver samples obtained from the same patient, and comparison with the ISG induction in matching PBMC samples originating from the same patient, allowed us to obtain unequivocal evidence that patients who respond poorly to the therapy show pre-activation of their IFN system, and that the pre-activation is confined to the liver and is not evident in PBMCs. Importantly, in patients with a low initial ISG expression, representing future responders to therapy, activation of the IFN system in response to pegIFNα did not exceed that seen in non-responders, either before or after therapy. This could suggest that patients with the initial pre-activation of the IFN system, future non-responders, have some defects at steps downstream of ISG expression, making them refractory to both endogenous IFN and IFN therapy.

IFNα treatment induced the STAT1 phosphorylation in all but one patient. There was a tendency for stronger STAT1 activation in RVR compared to non-RVR samples. However, the immunohistochemical analysis revealed a more pronounced difference. In non-RVR samples, pegIFNα strongly induced the nuclear STAT1 translocation in Kupffer cells, contrary to RVR samples, where nuclear STAT1 accumulation was induced predominantly in hepatocytes. Interestingly, non-RVR patients (with one exception) had nuclear phospho-STAT1 already present in pre-treatment biopsies. This is consistent with the observation that ISG transcripts are up-regulated in pre-treatment biopsies of later non-responders. How this preactivation of the Jak-STAT pathway is connected to the refractoriness of the IFN system in non-RVR patients requires further investigations.

Over the last few years, important insights into the interference of HCV with the innate immune system have been gained. Foremost, a series of elegant papers demonstrated the ability of HCV to inhibit both TLR3-TRIF-IRF3 and the RIG-I/MDA5-Cardif signaling pathways of IFNβ induction (27-33). This capacity of HCV could help to explain why the virus often establishes a chronic infection. However, our data and previously published results (20) demonstrate that the endogenous IFN system is constantly activated in many patients. Moreover, patients with a pre-activated IFN system seem to respond poorly to IFN therapy. This finding is counter-intuitive (one would expect that an active innate immune system would help to eliminate the virus during IFNα therapy), but it is largely supported by other published data from chimpanzees and human patients (16, 17, 20). From the analyses of ISG expression in liver biopsies it is apparent that in some patients HCV induces (or at least does not block) the endogenous IFN system, while in others it successfully represses it, possibly by cleaving TRIF and/or Cardif. Paradoxically, this difference has no apparent impact on the ability of HCV to maintain a chronic infection.

In patients without pre-activated IFN system, pegIFNα2b induced a robust up-regulation of many ISGs in the liver within 4 h. Similar high ISG expression was already present in the pre-treatment biopsies of patients that later did not show a rapid virological response at week 4. It is somewhat perplexing why the latter patients do not resolve the chronic HCV infection spontaneously despite the strong activation of the IFN system. One possibility is that ISG proteins that are up-regulated in both cases possess different post-transcriptional modifications. In an alternative scenario, non-response to both endogenous and exogenous IFNα may be caused by the lack of induction of a few critical ISGs that are specifically required for the elimination of HCV. We cannot exclude this possibility, but an array analysis performed on paired liver samples did not reveal ISGs that were specifically up-regulated in rapid responders. Furthermore, this model cannot explain why pre-activation of the endogenous IFN system is so closely linked to later non-response to treatment.

Alternatively, the kinetics of induction of the interferon response could be decisive. In the patients without pre-activated IFN system, the injection of exogenous IFNα during treatment should induce an antiviral state very rapidly in most liver cells, and HCV would not have “enough” time to escape from the IFN-induced defense. On the other hand, the build-up of the antiviral state could be slow in the other group of patients which would give HCV enough time to adapt to and evade the intracellular antiviral defense system, making it also resistant to the subsequent IFN therapy.

How could the induction of the endogenous IFN system compromise the success of IFNα therapy? Clearly, the activation of negative feedback loops that inhibit IFN signaling could play a role. Prominent candidates amongst the negative regulators are: suppressors of cytokine signaling 1 (SOCS1) and SOCS3 (34), two IFN induced proteins that bind to the IFN receptor and inhibit the activity of Jak1 and Tyk2; and the more recently described regulator Ubp43, an IFN-stimulated protein that binds to IFNα receptor 2 (IFNAR2) and blocks the access of Jak1 to it (35). However, we could not find a significant difference in the expression levels of these negative regulators in the pegIFNα2b stimulated liver biopsies of RVR compared to non-RVR patients (data not shown). Moreover, a general up-regulation of negative regulators such as SOCSs and Ubp43 is not compatible with the observed strong constitutive expression of a large number of ISGs in the subset of patients that poorly respond to IFN therapy. If IFNα signaling were indeed inhibited by the induction of SOCSs and Ubp43 in the majority of liver cells, then one should not observe such a pronounced pre-activation of ISGs in pre-treatment livers.

Notably, the pre-activation of tested ISGs occurred more frequently in liver biopsies of patients infected with HCV genotype 1 and 4 than with genotypes 2 or 3. It is well known that genotype 2 and 3 infections can be cured in over 80% of patients, compared to less than 50% of infections with genotype 1 (4). Our finding that the frequency and degree of pre-activation of the endogenous IFN system depends on the HCV genotype could provide an explanation for this differential susceptibility. Perhaps HCV genotypes 2 and 3 are more successful in preventing the activation of innate immunity in the liver by a more effective cleavage of Cardif and/or TRIF. The success of the virus in preventing the induction of the endogenous IFN system would however come at the cost of being more susceptible to IFNα therapies. Of note, a single chimpanzee infected with the genotype 3 HCV has been shown to have lower ISG expression levels than animals infected with genotype 1 (17).

We have shown previously that HCV inhibits the IFNα-induced signaling via the Jak-STAT pathway by up-regulating a protein phosphatase PP2A (12, 14, 25, 36). PP2A is a heterotrimeric complex of a scaffolding A, a regulatory B, and a catalytic C subunits. The PP2Ac subunit expression is significantly higher in livers of patients infected with genotype 1 than genotype 3 (25). As shown in this work, the expression of PP2Ac mRNA is higher in biopsies of later non-responders than responders. These data support a model where HCV interference with the IFN signaling impairs the response to therapy. Moreover, inhibition of the IFNα signaling by HCV could also explain why the strong pre-activation of the endogenous IFN system does not lead to a spontaneous elimination of HCV. If one assumes that not all hepatocytes are infected by HCV, but rather a minority, then the induction of ISGs observed in pre-treatment biopsies of non-RVR patients could occur predominantly in non-infected hepatocytes. In the infected cells, IFN would be ineffective because of the inhibition of the Jak-STAT signaling pathway. The IFN responsible for the pre-activation of the system would be secreted by hepatocytes that are infected with a virus that is not successful in cleaving Cardif and/or TRIF. Because of the HCV-induced inhibition of the Jak-STAT pathway, the secreted IFNβ would not induce an antiviral state in the infected hepatocytes, but rather in non-infected neighbor cells. To gain further insights into the pathobiology of CHC, future studies should focus on analysis at the single-cell level. Unfortunately, the detection of HCV infected hepatocytes in liver biopsies is still unsatisfactory, making such studies difficult.

Although the precise mechanism of the HCV escape from the immune defense system still remains to be elucidated, the impairment of the hepatitis C therapy by pre-activation of the endogenous IFN system is now well established. It would be interesting to investigate if this pre-activation is a reversible process. The injection of neutralizing anti-IFNα/β antibodies or other factors blocking the IFN response before treatment could return the endogenous IFN system to a “naive” state, and potentially enhance the response to IFNα based therapies.

TABLE 1

| | | | | | Baseline | 4 week | 12 week | | | |
|---|---|---|---|---|---|---|---|---|---|---|
| | | | | | VL | VL | VL | | | |
| Patient | 4 week | Sex | Age | HCV | (log | (log | (log | 12 week | Metavir | Weight |
| Nr. | response | (m/f) | (years) | genotype | IU/ml) | IU/ml) | IU/ml) | response | (grade/stage) | (kg) |
| 1 | RVR | m | 52 | 3a | 7.14 | neg. | neg. | EVR | A2/F2 | 75 |
| 2 | RVR | m | 37 | 3a | 4.90 | neg. | neg. | EVR | A1/F2 | 73 |
| 3 | RVR | m | 38 | 1a | 6.91 | neg. | neg. | EVR | A2/F1 | 85 |
| 4 | RVR | m | 33 | 2b | 6.27 | neg. | neg. | EVR | A1/F2 | 57 |
| 5 | RVR | m | 48 | 2b | 6.67 | neg. | neg. | EVR | A3/F4 | 110 |
| 6 | RVR | f | 53 | 2a/c | 4.95 | neg. | neg. | EVR | A3/F3 | 74 |
| 7 | Non- | f | 54 | 3a | 4.52 | 4.08 | 1.3 | EVR | A3/F4 | 69 |
| | RVR | | | | | | | | | |
| 8 | Non- | m | 64 | 1b | 6.24 | 4.83 | 3.46 | EVR | A3/F4 | 74 |
| | RVR | | | | | | | | | |
| 9 | Non- | m | 56 | 1b | 6.89 | 6.76 | 6.01 | PNR | A2/F3 | 60 |
| | RVR | | | | | | | | | |
| 10 | Non- | f | 50 | 1a | 7.11 | 6.58 | 6.35 | PNR | A1/F2 | 77 |
| | RVR | | | | | | | | | |
| 11 | Non- | f | 47 | 1a | 6.16 | 5.99 | 5.52 | PNR | A2/F2 | 81 |
| | RVR | | | | | | | | | |

TABLE 2
Analysis of gene expression in pre-treatment biopsies (B-1). List of 83 genes best predicting treatment outcome at week 4 (IFN regulated genes shaded in grey, up-regulated genes in bold, down-regulated genes italic; genes that differ between RVR and non-RVR but are not regulated by IFN are not shaded).

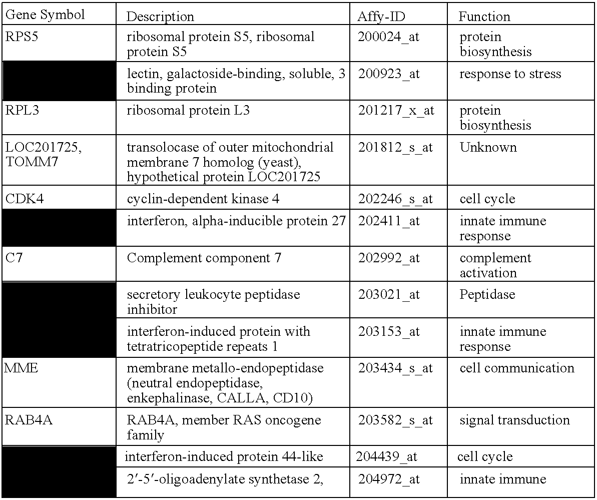

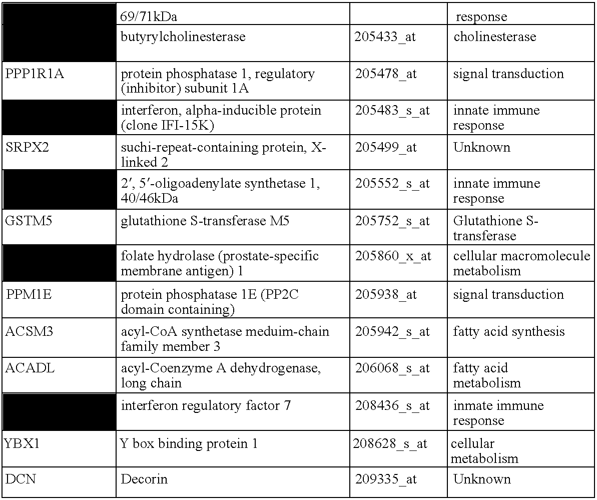

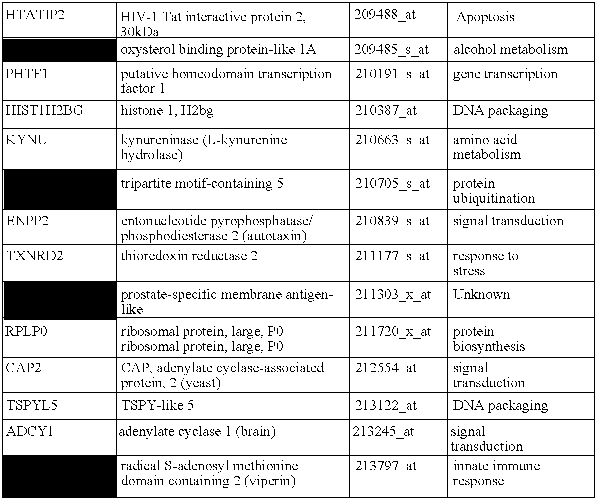

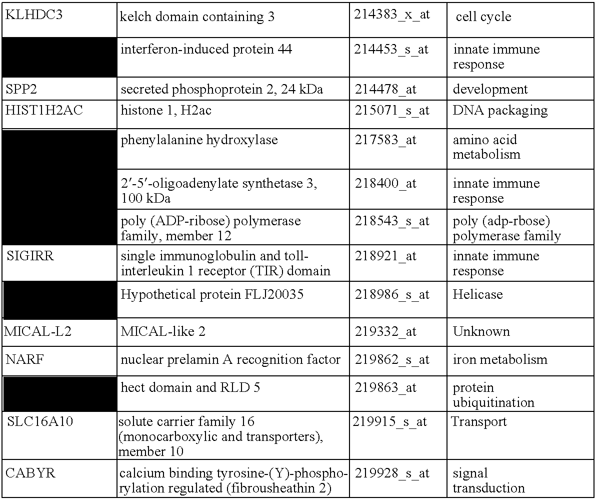

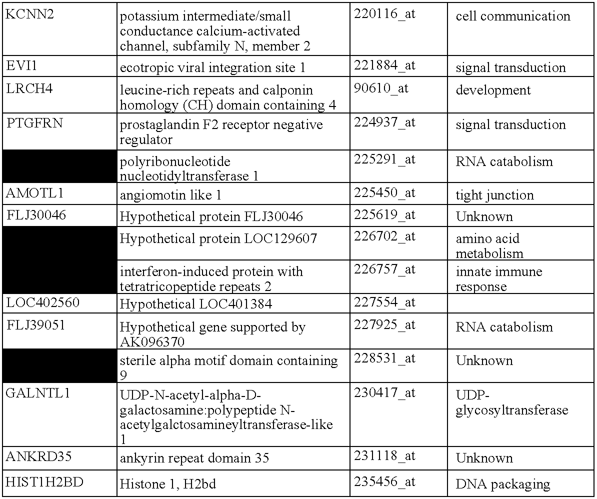

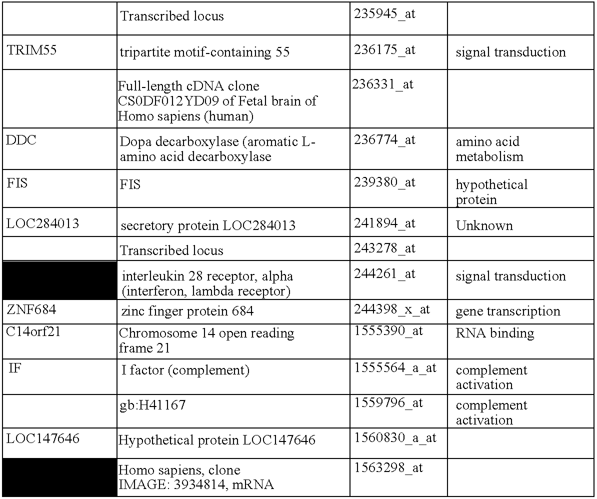

TABLE 3
Analysis of gene expression in biopsies obtained 4 hours after pegIFNα
(B-2). List of 173 genes best predicting treatment outcome at week 4
(IFN regulated genes shaded in grey, up-regulated genes in bold,
down-regulated genes italic; genes that differ between RVR and
non-RVR but are not regulated by IFN are not shaded).

| Gene | | |
|---|---|---|
| Symbol | Description | Affy-ID |
| ACSL3 | acyl-CoA synthetase long-chain family | 201662_s_at |
| | member 3 | |
| ACSM3 | acyl-CoA synthetase medium-chain | 205942_s_at |
| | family member 3 | |
| ADCY1 | adenylate cyclase 1 (brain) | 213245_at |
| ADRB2 | adrenergic, beta-2-, receptor, surface | 206170_at |
| AMOTL1 | angiomotin like 1 | 225450_at |
| ANXA10 | annexin A10 | 210143_at |
| ARHGEF16 | Rho guanine exchange factor (GEF) | 208009_s_at |
| | 16 | |
| ATP2A2 | ATPase, Ca++ transporting, cardiac | 209186_at |
| | muscle, slow twitch 2 | |
| BCHE | butyrylcholinesterase | 205433_at |
| BRUNOL5 | bruno-like 5, RNA binding protein | 230497_at |
| | (Drosophila) | |
| C10orf125 | chromosome 10 open reading frame | 230259_at |
| | 125 | |
| 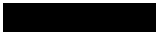 | chromosome 1 open reading frame 96 | 225904_at |
| C21orf106 | chromosome 21 open reading frame | 1561286_a_at |
| | 106 | |
| C22orf3 | chromosome 22 open reading frame 3 | 217622_at |
| C3orf4 | chromosome 3 open reading frame 4 | 239146_at |
| C6orf7l | chromosome 6 open reading frame 71 | 231070_at |
| C8orf47 | chromosome 8 open reading frame 47 | 1552389_at |
| C9orf95 | chromosome 9 open reading frame 95 | 219147_s_at |
| CAMK2D | calcium/calmodulin-dependent protein | 225019_at |
| | kinase (CaM kinase) II delta | |
| CAPN3 | calpain 3, (p94) | 211890_x_at |
| CCL14, | chemokine (C-C motif) ligand 14, | 210390_s_at |
| CCL15 | chemokine (C-C motif) ligand 15 | |
| CCT5 | chaperonin containing TCP1, subunit | 208696_at |
| | 5 (epsilon) | |
| CDKN2A | cyclin-dependent kinase inhibitor 2A | 207039_at |
| | (melanoma, p16, inhibits CDK4) | |
| CES7 | carboxylesterase 7 | 1553465_a_at |
| CFHL5 | complement factor H-related 5, | 208088_s_at |
| | complement factor H-related 5 | |
| CHMP4A, | chromatin modifying protein 4A, | 228764_s_at |
| MGC5987 | hypothetical protein MGC5987 | |
| CNDP1 | carnosine dipeptidase 1 | 223699_at |
| | (metallopeptidase M20 family) | |
| CTNNA1 | catenin (cadherin-associated protein), | 210844_x_at |
| | alpha 1, 102 kDa | |
| CUEDC1 | CUE domain containing 1 | 219468_s_at |
| 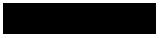 | chemokine (C-X-C motif) ligand 2 | 209774_x_at |
| CYP11A1 | cytochrome P450, family 11, | 204309_at |
| | subfamily A, polypeptide 1, | |
| | cytochrome P450, family 11, | |
| | subfamily A, polypeptide 1 | |
| DAAM1 | Dishevelled associated activator of | 1555989_at |
| | morphogenesis 1 | |
| DBT | Dihydrolipoamide branched chain | 231919_at |
| | transacylase E2 | |
| DHRS10 | dehydrogenase/reductase (SDR | 228713_s_at |
| | family) member 10 | |
| DPAGT1 | dolichyl-phosphate (UDP-N- | 209509_s_at |
| | acetylglucosamine) N- | |
| | acetylglucosaminephosphotransferase | |
| | 1 (GlcNAc-1-P transferase) | |
| EEF1G | eukaryotic translation elongation | 200689_x_at |
| | factor 1 gamma | |
| EFHD1 | EF-hand domain family, member D1 | 209343_at |
| ENPP2 | ectonucleotide | 210839_s_at |
| | pyrophosphatase/phosphodiesterase | |
| | 2 (autotaxin) | |
| ESR1 | estrogen receptor 1 | 205225_at |
| EXOC4 | Exocyst complex component 4 | 1557772_at |
| FIS | FIS | 239380_at |
| FLJ21511 | hypothetical protein FLJ21511 | 220723_s_at |
| FLJ34790 | hypothetical protein FLJ34790 | 230012_at |
| 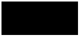 | folate hydrolase (prostate-specific membrane antigen) 1 | 205860_x_at |
| FZD3 | frizzled homolog 3 (Drosophila) | 219683_at |
| GLRX2 | glutaredoxin 2 | 219933_at |
| GPC3 | glypican 3 | 209220_at |
| GPR143 | G protein-coupled receptor 143 | 206696_at |
| HCG4 | HLA complex group 4 | 206685_at |
| HERC4 | hect domain and RLD 4 | 225988_at |
| HIST1H2AC | histone 1, H2ac | 215071_s_at |
| HKDC1 | hexokinase domain containing 1 | 227614_at |
| 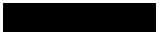 | H2.0-like homeo box 1 (Drosophila) | 214438_at |
| HLXB9 | homeo box HB9 | 214614_at |
| HSPA5 | heat shock 70 kDa protein 5 (glucose- | 211936_at |
| | regulated protein, 78 kDa) | |
| HTATIP2 | HIV-1 Tat interactive protein 2, 30 kDa | 209448_at |
| HYI | hydroxypyruvate isomerase homolog | 221435_x_at |
| | (E. coli), hydroxypyruvate isomerase | |
| | homolog (E. coli) | |
| IDH3A | isocitrate dehydrogenase 3 (NAD+) | 202070_s_at |
| | alpha | |
| IDS | iduronate 2-sulfatase (Hunter | 206342_x_at |
| | syndrome) | |
| IFI27 | interferon, alpha-inducible protein 27 | 202411_at |
| ITGA6 | integrin, alpha 6 | 201656_at |
| KCNN2 | potassium intermediate/small | 220116_at |
| | conductance calcium-activated | |
| | channel, subfamily N, member 2 | |
| KIAA0090 | KIAA0090 | 212395 s_at |
| KIAA0446 | KIAA0446 gene product | 212683_at |
| KIAA0888 | KIAA0888 protein | 235048_at |
| KIAA1522 | KIAA1522 | 224746_at |
| KIAA1609 | KIAA1609 protein | 65438_at |
| KIAA2002 | KIAA2002 protein | 225913_at |
| KPNA2 | karyopherin alpha 2 (RAG cohort 1, | 211762_s_at |
| | importin alpha 1), karyopherin alpha 2 | |
| | (RAG cohort 1, importin alpha 1) | |
| LGALS3BP | lectin, galactoside-binding, soluble, 3 | 200923_at |
| | binding protein | |
| LOC201725, | translocase of outer mitochondrial | 201812_s_at |
| TOMM7 | membrane 7 homolog (yeast), | |
| | hypothetical protein LOC201725 | |
| LOC202775, | hypothetical LOC202775, hypothetical | 241353_s_at |
| LOC402617 | LOC402617 | |
| LOC221710 | hypothetical protein LOC221710 | 1564651_at |
| LOC286434, | hypothetical protein LOC286434, | 222031_at |
| LOC389906, | similar to Serine/threonine-protein | |
| LOC389908 | kinase PRKX (Protein kinase PKX1), | |
| | hypothetical LOC389908 | |
| LOC402560 | Hypothetical LOC401384 | 227554_at |
| LOC90268 | hypothetical protein BC007706 | 229268_at |
| LONRF1 | LON peptidase N-terminal domain | 226038_at |
| | and ring finger 1 | |
| LRRC20 | leucine rich repeat containing 20 | 218550_s_at |
| MAFB | v-maf musculoaponeurotic | 218559_s_at |
| | fibrosarcoma oncogene homolog B | |
| | (avian) | |
| MDK | midkine (neurite growth-promoting | 209035_at |
| | factor 2) | |
| MEF2D | MADS box transcription enhancer | 203003_at |
| | factor 2, polypeptide D (myocyte | |
| | enhancer factor 2D) | |
| MEGF10 | MEGF10 protein | 232523_at |
| MFN1 | mitofusin 1 | 217043_s_at |
| MGC39545 | hypothetical protein LOC403312 | 1555002_at |
| MICAL-L2 | MICAL-like 2 | 219332_at |
| MME | membrane metallo-endopeptidase | 203434_s_at |
| | (neutral endopeptidase, | |
| | enkephalinase, CALLA, CD10) | |
| MORC4 | MORC family CW-type zinc finger 4 | 219038_at |
| MRAP | melanocortin 2 receptor accessory | 1555741_at |
| | protein | |
| MYH14 | myosin, heavy polypeptide 14 | 234290_x_at |
| NEU4 | sialidase 4 | 222957_at |
| NFASC | Neurofascin | 213438_at |
| OAT | ornithine aminotransferase (gyrate | 201599_at |
| | atrophy) | |
| OSTalpha | organic solute transporter alpha | 229230_at |
| PAPPA2 | Pappalysin 2 | 213332_at |
| PARP6 | poly (ADP-ribose) polymerase family, | 219639_x_at |
| | member 6 | |
| PBX 1 | Pre-B-cell leukemia transcription | 1562235_s_at |
| | factor 1 | |
| PCBD2 | 6-pyruvoyl-tetrahydropterin | 223712_at |
| | synthase/dimerization cofactor of | |
| | hepatocyte nuclear factor 1 alpha | |
| | (TCF1) 2 | |
| PCOLCE | procollagen C-endopeptidase | 202465_at |
| | enhancer | |
| PCYOX1 | prenylcysteine oxidase 1 | 225274_at |
| PDE4DIP | phosphodiesterase 4D interacting | 210305_at |
| | protein (myomegalin) | |
| PPAPDC2 | phosphatidic acid phosphatase type 2 | 227385_at |
| | domain containing 2 | |
| PPARD | peroxisome proliferative activated | 242218_at |
| | receptor, delta | |
| PPGB | protective protein for beta- | 200661_at |
| | galactosidase (galactosialidosis) | |
| PPID | Peptidylprolyl isomerase D (cyclophilin | 228469_at |
| | D) | |
| PPP1R1A | protein phosphatase 1, regulatory | 205478_at |
| | (inhibitor) subunit 1A | |
| PPP2R1B | protein phosphatase 2 (formerly 2A), | 222351_at |
| | regulatory subunit A (PR 65), beta | |
| | isoform | |
| PSMAL | prostate-specific membrane antigen- | 211303_x_at |
| | like | |
| PTGFRN | prostaglandin F2 receptor negative | 224950_at |
| | regulator | |
| RAB11FIP5 | RAB11 family interacting protein 5 | 210879_s_at |
| | (class I) | |
| RABEPK | Rab9 effector protein with kelch motifs | 1558021_at |
| RAD52 | RAD52 homolog (S. cerevisiae), | 211994_at |
| | RAD52 homolog (S. cerevisiae) | |
| RAI14 | retinoic acid induced 14 | 202052_s_at |
| RPL14, | ribosomal protein L14, ribosomal | 200074_s_at |
| RPL14L | protein L14, ribosomal protein L14- | |
| | like, ribosomal protein L14-like | |
| RPL3 | ribosomal protein L3, ribosomal | 211666_x_at |
| | protein L3 | |
| RPLP0 | ribosomal protein, large, P0 | 201033_x_at |
| RPLP0 | ribosomal protein, large, P0 | 211972_x_at |
| RPS5 | ribosomal protein S5, ribosomal | 200024_at |
| | protein S5 | |
| RRBP1 | ribosome binding protein 1 homolog | 201206_s_at |
| | 180 kDa (dog) | |
| SAC3D1 | SAC3 domain containing 1 | 205449_at |
| SDCBP2 | syndecan binding protein (syntenin) 2 | 233565_s_at |
| SEMA4F | sema domain, immunoglobulin | 210124_x_at |
| | domain (Ig), transmembrane domain | |
| | (TM) and short cytoplasmic domain, | |
| | (semaphorin) 4F | |
| SERPINA7 | serpin peptidase inhibitor, clade A | 206386_at |
| | (alpha-1 antiproteinase, antitrypsin), | |
| | member 7 | |
| SET7 | SET domain-containing protein 7 | 224928_at |
| SH3MD1 | SH3 multiple domains 1 | 224817_at |
| SHD | Src homology 2 domain containing | 227845_s_at |
| | transforming protein D | |
| SIGIRR | single immunoglobulin and toll- | 52940_at |
| | interleukin 1 receptor (TIR) domain | |
| SLC16A10 | solute carrier family 16 | 219915_s_at |
| | (monocarboxylic acid transporters), | |
| | member 10 | |
| SLC16A4 | solute carrier family 16 | 205234_at |
| | (monocarboxylic acid transporters), | |
| | member 4 | |
| SLC19A2 | solute carrier family 19 (thiamine | 209681_at |
| | transporter), member 2 | |
| 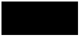 | solute carrier family 23 (nucleobase transporters), member 2 | 209236_at |
| SLC25A16 | Solute carrier family 25 (mitochondrial | 235747_at |
| | carrier; Graves disease autoantigen), | |
| | member 16 | |
| SLC25A37 | solute carrier family 25, member 37 | 218136_s_at |
| SLC6A8 | solute carrier family 6 | 210854_x_at |
| | (neurotransmitter transporter, | |
| | creatine), member 8 | |
| SLP1 | secretory leukocyte peptidase inhibitor | 203021_at |
| SMBP | SM-11044 binding protein | 217758_s_at |
| SNF1LK2 | SNF1-like kinase 2 | 213221_s_at |
| SPP2 | secreted phosphoprotein 2, 24 kDa | 214478_at |
| SSTR1 | somatostatin receptor 1 | 235591_at |
| ST13 | suppression of tumorigenicity 13 | 208667_s_at |
| | (colon carcinoma)(Hsp70 interacting | |
| | protein) | |
| ST7L | suppression of tumorigenicity 7 like | 1552739_s_at |
| STMN1 | Stathmin 1/oncoprotein 18 | 217253_at |
| STS | steroid sulfatase (microsomal), | 203768_s_at |
| | arylsulfatase C, isozyme S | |
| TM4SF4 | transmembrane 4 L six family | 209937_at |
| | member 4 | |
| TMED10 | transmembrane emp24-like trafficking | 200929_at |
| | protein 10 (yeast) | |
| TRIM55 | tripartite motif-containing 55 | 236175_at |
| UBE1L | ubiquitin-activating enzyme E1-like | 203281_s_at |
| VISA | Virus-induced signaling adapter | 229741_at |
| VKORC1L1 | vitamin K epoxide reductase complex, | 224881_at |
| | subunit 1-like 1 | |
| WWOX | WW domain containing | 210695_s_at |
| | oxidoreductase | |
| WWOX | WW domain containing | 219077_s_at |
| | oxidoreductase | |
| YLPM1 | YLP motif containing 1 | 214659_x_at |
| ZD52F10 | Dermokine | 226926_at |
| ZFP3 | zinc finger protein 3 homolog (mouse) | 235728_at |
| ZNF511 | zinc finger protein 511 | 225307_at |
| ZNF710 | Zinc finger protein 710 | 213658_at |
| | CDNA FLJ41000 fis, clone | 201204_s_at |
| | UTERU2016761, highly similar to | |
| | Homo sapiens ES/130 mRNA | |
| | gb:AI341383/DB_XREF = gi:4078310 | 227092_at |
| | /DB_XREF = qx91a06.x1 | |
| | /CLONE = IMAGE:2009842 /FEA = EST | |
| | /CNT = 52 /TID = Hs.112751.2 | |
| | /TIER = Stack /STK = 42 | |
| | /UG = Hs.112751 /LL = 23383 | |
| | /UG_GENE = KIAA0892 | |
| | /UG_TITLE = KIAA0892 protein | |
| | CDNA FLJ38567 fis, clone | 228240_at |
| | HCHON2005166 | |
| | CDNA: FLJ21462 fis, clone | 228732_at |
| | COL04744 | |
| | gb:AI357655 /DB_XREF = gi:4109276 | 228971_at |
| | /DB_XREF = qy15d03.x1 | |
| | /CLONE = IMAGE:2012069 /FEA = EST | |
| | /CNT = 15 /TID = Hs.292931.0 | |
| | /TIER =Stack /STK = 13 | |
| | /UG = Hs.292931 /UG_TITLE = ESTs | |
| | gb:AU144136 | 233799_at |
| | /DB_XREF = gi:11005657 | |
| | /DB_XREF = AU144136 | |
| | /CLONE = HEMBA1000972 | |
| | /FEA = mRNA /CNT = 5 | |
| | /TID = Hs.296647.0 /TIER = ConsEnd | |
| | /STK = 2 /UG = Hs.296647 | |
| | /UG_TITLE = Homo sapiens cDNA | |
| | FLJ11418 fis, clone HEMBA1000972 | |
| | CDNA FLJ41376 fis, clone | 235133_at |
| | BRCAN2008494 | |
| | gb:AW167727 | 235201_at |
| | /DB_XREF = gi:6399252 | |
| | /DB_XREF = xn48c05.x1 | |
| | /CLONE = IMAGE:2696936 /FEA = EST | |
| | /CNT = 15 /TID = Hs.11873.0 | |
| | /TIER = ConsEnd /STK = 3 | |
| | /UG = Hs.11873 /UG_TITLE = ESTs | |
| | Transcribed locus, strongly similar to | 235651_at |
| | XP_513428.1 PREDICTED: similar to | |
| | hypothetical protein 4732467L 16 [Pan | |
| | troglodytes] | |
| | Transcribed locus | 235945_at |
| | Transcribed locus | 236220_at |
| | gb:BF437161 | 239082_at |
| | /DB_XREF = gi:11449494 | |
| | /DB_XREF7 = 7p67c05.xl | |
| | /CLONE = IMAGE:3650697 /FEA = EST | |
| | /CNT = 6 /TID = Hs.208261.0 | |
| | /TIER = ConsEnd /STK = 4 | |
| | /UG = Hs.208261 /UG_TITLE = ESTs | |
| | gb:N57929 /DB_XREF = gi:1201819 | 242978_x_at |
| | /DB_XREF = yv6le06.s1 | |
| | /CLONE = IMAGE:247234 /FEA = EST | |
| | /CNT = 4 /TID = Hs.48100.0 | |
| | /TIER = ConsEnd /STK = 3 | |
| | /UG = Hs.48100 /UG_TITLE = ESTs | |
| | Transcribed locus | 243130_at |
| | Transcribed locus | 243278_at |
| | Transcribed locus | 243302_at |
| | gb:H41167 /DB_XREF = gi:917219 | 1559776_at |
| | /DB_XREF = yp64g06.s1 | |
| | /CLONE = IMAGE:192250 | |
| | /TID = Hs2.191285.1 /CNT = 4 | |
| | /FEA = mRNA /TIER = ConsEnd | |
| | /STK = 0 | |
| | /UG = Hs.191285 /UG_TITLE = Homo | |
| | sapiens cDNA FLJ36989 fis, clone | |
| | BRACE2006753. | |

TABLE 4
Primer sequences used for
real-time RT-PCR analysis.

| Gene | Primer 1 sequence | Primer 2 sequence |
|---|---|---|
| GAPDH | 5′ GCTCCTCCTGTTCGACAGT | 5′ ACCTTCCCCATGGTG TC |
| | CA 3′ | TGA 3′ |
| STAT1 | 5′ TCCCCA GGCCCTTGTT | 5′ CAAGCTGCTGAAGTTGGT |
| | G 3′ | ACCA 3′ |
| IP10 | 5′ CGATTCTGATTTGCTGCCT | 5′ GCAGGTACAGCGTACGGT |
| | TAT 3′ | TCT 3′ |
| SOCS1 | 5′ CCCCTTCTGTAGGATGGTA | 5′ TGCTGT GGAGACTGCAT |
| | GCA 3′ | TGTC 3′ |
| SOCS3 | 5′ TGCGCCTCAAGACCT TCA | 5′ CTTGCGCACTGCGTTCA |
| | G 3′ | C 3′ |
| USP18 | 5′ CTC AGTCCCGACGTGGAA | 5′ ATCTCTCAAGCGCCATGC |
| | CT 3′ | A 3′ |
| IF127 | 5′ CCTCGGGCAGCCTTGT | 5′ AATCCGGAGAGTCCAGTT |
| | G 3′ | GCT 3′ |

### (c) 1.4 References

1. Li, K., et al. Immune evasion by hepatitis C virus NS3/4A protease-mediated cleavage of the Toll-like receptor 3 adaptor protein TRIF. Proc Natl Acad Sci USA 102, 2992-2997 (2005).
2. Yoneyama, M., et al. The RNA helicase RIG-I has an essential function in double-stranded RNA-induced innate antiviral responses. Nat Immunol 5, 730-737 (2004).
3. Kang, D. C., et al. mda-5: An interferon-inducible putative RNA helicase with double-stranded RNA-dependent ATPase activity and melanoma growth-suppressive properties. Proc Natl Aced Sci USA 99, 637-642 (2002).
4. Kawai, T., at al. IPS-1, an adaptor triggering RIG-I- and Mda5-mediated type I interferon induction. Nat Immunol 6, 981-988 (2005).
5. Meylan, E., et al. Cardif is an adaptor protein in the RIG-1 antiviral pathway and is targeted by hepatitis C virus. Nature 437, 1167-1172 (2005).
6. Seth, R. B., Sun, L., Ea, C. K. & Chen, Z. J. Identification and characterization of MAVS, a mitochondrial antiviral signaling protein that activates NF-kappaB and IRF 3. Cell 122, 669-682 (2005).
7. Xu, L. G., et al. VISA is an adapter protein required for virus-triggered IFN-beta signaling. Mol Cell 19, 727-740 (2005).
8. Gale, M., Jr. & Foy, E. M. Evasion of intracellular host defence by hepatitis C virus. Nature 436, 939-945 (2005).
9. Bigger, C. B., Brasky, K. M. & Lanford, R. E. DNA microarray analysis of chimpanzee liver during acute resolving hepatitis C virus infection. J Virol 75, 7059-7066 (2001).
10. Bigger, C. B., et al. Intrahepatic gene expression during chronic hepatitis C virus infection in chimpanzees. J Virol 78, 13779-13792 (2004).
11. Chen, L., et al. Hepatic gene expression discriminates responders and nonresponders in treatment of chronic hepatitis C viral infection. Gastroenterology 128, 1437-1444 (2005).
12. Strader, D. B., Wright, T., Thomas, D. L. & Seeff, L. B. Diagnosis, management, and treatment of hepatitis C. Hepatology 39, 1147-1171 (2004).
13. Manns, M. P., et al. Peginterferon alfa-2b plus ribavirin compared with interferon alfa-2b plus ribavirin for initial treatment of chronic hepatitis C: a randomised trial. Lancet 358, 958-965 (2001).
14. Fried, M. W., et al. Peginterferon alfa-2a plus ribavirin for chronic hepatitis C virus infection. N Engl J Med 347, 975-982 (2002).
15. Hadziyannis, S. J., et al. Peginterferon-alpha2a and ribavirin combination therapy in chronic hepatitis C: a randomized study of treatment duration and ribavirin dose. Ann Intern Med 140, 346-355 (2004).
16. Lau, D. T., et al. 10-Year follow-up after interferon-alpha therapy for chronic hepatitis C. Hepatology 28, 1121-1127 (1998).
17. Marcellin, P., et al. Long-term histologic improvement and loss of detectable intrahepatic HCV RNA in subjects with chronic hepatitis C and sustained response to interferon-alpha therapy. Ann Intern Med 127, 875-881 (1997).
18. Ferenci, P., et al. Predicting sustained virological responses in chronic hepatitis C subjects treated with peginterferon alfa-2a (40 KD)/ribavirin. J Hepatol 43, 425-433 (2005).
19. Davis, G. L., et al. Early virologic response to treatment with peg interferon alfa-2b plus ribavirin in subjects with chronic hepatitis C. Hepatology 38, 645-652 (2003).
20. Yu, J. W., Wang, G. Q., Sun, L. J., Li, X. G. & Li, S. C. Predictive value of rapid virological response and early virological response on sustained virological response in HCV subjects treated with pegylated interferon alpha-2a and ribavirin. Journal of gastroenterology and hepatology 22, 832-836 (2007).
21. Sen, G. C. Viruses and interferons. Annual review of microbiology 55, 255-281 (2001).
22. Darnell, J. E., Jr., Kerr, I. M. & Stark, G. R. Jak-STAT pathways and transcriptional activation in response to IFNs and other extracellular signaling proteins. Science 264, 1415-1421 (1994).
23. de Veer, M. J., et al. Functional classification of interferon-stimulated genes identified using microarrays. Journal of leukocyte biology 69, 912-920 (2001).
24. Blindenbacher, A., et al. Expression of hepatitis c virus proteins inhibits interferon alpha signaling in the liver of transgenic mice. Gastroenterology 124, 1465-1475 (2003).
25. Heim, M. H., Moradpour, D. & Blum, H. E. Expression of hepatitis C virus proteins inhibits signal transduction through the Jak-STAT pathway. J Virol 73, 8469-8475 (1999).
26. Duong, F. H., Filipowicz, M., Tripodi, M., La Monica, N. & Heim, M. H. Hepatitis C virus inhibits interferon signaling through up-regulation of protein phosphatase 2A. Gastroenterology 126, 263-277 (2004).
27. Gale, M., Jr., et al. Control of PKR protein kinase by hepatitis C virus nonstructural 5A protein: molecular mechanisms of kinase regulation. Mol Cell Biol 18, 5208-5218 (1998).
28. Wang, C., et al. Alpha interferon induces distinct translational control programs to suppress hepatitis C virus RNA replication. J Virol 77, 3898-3912 (2003).
29. Taylor, D. R., Shi, S. T., Romano, P. R., Barber, G. N. & Lai, M. M. Inhibition of the interferon-inducible protein kinase PKR by HCV E2 protein. Science 285, 107-110 (1999).
30. Silva, M., et al. A randomised trial to compare the pharmacokinetic, pharmacodynamic, and antiviral effects of peg interferon alfa-2b and peg interferon alfa-2a in subjects with chronic hepatitis C (COMPARE). J Hepatol 45, 204-213 (2006).
31. Shuai, K., Stark, G. R., Kerr, I. M. & Darnell, J. E., Jr. A single phosphotyrosine residue of Stat91 required for gene activation by interferon-gamma. Science 261, 1744-1746 (1993).
32. Darnell, J. E., Jr. STATs and gene regulation. Science 277, 1630-1635 (1997).
33. Duong, F. H., Christen, V., Filipowicz, M. & Heim, M. H. S-adenosylmethionine and betaine correct hepatitis C virus induced inhibition of interferon signaling in vitro. Hepatology 43, 796-806 (2006).
34. Lanford, R. E., et al. Genomic response to interferon-alpha in chimpanzees: implications of rapid downregulation for hepatitis C kinetics. Hepatology 43, 961-972 (2006).
35. Krebs, D. L. & Hilton, D. J. SOCS proteins: negative regulators of cytokine signaling. Stem Cells 19, 378-387 (2001).
36. Malakhova, O. A., et al. UBP43 is a novel regulator of interferon signaling independent of its ISG15 isopeptidase activity. Embo J 25, 2358-2367 (2006).
37. McHutchison, J. G., et al. Interferon alfa-2b alone or in combination with ribavirin as initial treatment for chronic hepatitis C. Hepatitis Interventional Therapy Group. N Engl J Med 339, 1485-1492 (1998).
38. Christen, V., Treves, S., Duong, F. H. & Heim, M. H. Activation of endoplasmic reticulum stress response by hepatitis viruses up-regulates protein phosphatase 2A. Hepatology (2007).

1-20. (canceled)

21. A method for determining the likelihood that a subject having a viral infection of the liver will be responsive to antiviral therapy that includes stimulation of Interferon (IFN) activity, the method comprising:
(a) analysing a sample from the subject for expression of at least one gene from each of the following groups of genes:
(i) KYNU; PAH; LOC129607; DDC; FOLH1; YBX1; BCHE; ACADL; ACSM3; NARF; SLPI; RPS5; RPL3; RPLP0; TRIM5 and HERC5;
(ii) HTATIP2; CDK4; IFI44L; and KLHDC3;
(iii) C7; IF; IF127; IFIT1; OAS2; G1P2; OAS1; IRF7; RSAD2; IF144; OAS3; SIGIRR; and IFIT2;
(iv) RAB4A; PPP1R1A; PPM1E; ENPP2; CAP2; ADCY1; CABYR; EVI1; PTGFRN;
TRIM55; and IL28RA;
(v) MME; KCNN2; SLC16A10; AMOTL1; SPP2; LRCH4; HIST1H2BG; TSPYL5; HIST1H2AC; HIST1H2BD; PHTF1; ZNF684; GSTM5; FLJ20035; FIS; PARP12; C14orf21; PNPT1; FLJ39051; GALNTL1; OSBPL1A; LGALS3BP; TXNRD2; LOC201725, TOMM7; SRPX2; DCN; PSMAL; MICAL-L2; FLJ30046; SAMD9; ANKRD35; LOC284013; LOC402560; and LOC147646; and,
(b) comparing expression of the genes in the sample from the subject to expression of the same genes in a control sample, wherein an increase in the level of expression of the genes in the sample from the subject before said antiviral therapy relative to the level of expression of the same genes in the control sample indicates that the subject is not likely to be responsive to said antiviral therapy.

22. The method of claim 1 wherein the antiviral therapy includes pegylated IFNα.

23. The method of claim 2 wherein the antiviral therapy further includes ribavirin.

24. The method of claim 1 wherein the viral infection is Hepatitis B virus or Hepatitis C virus infection.

25. The method of claim 4 wherein the virus is Hepatitis C virus.

26. The method of claim 1 wherein the sample comprises liver tissue.

27. The method of claim 1 wherein the expression of five, six, seven, eight, nine, 10, 11, 12, 13, 14, 15, 20, 25, 30, 35, 40, 45, 50, 55, 60, 65, 70, 71, 72, 73, 74, 75, 76, 77 or 78 genes is analysed.

28. The method of claim 1 wherein gene expression is determined by measuring the amount of mRNA gene transcript in the sample, or the amount of cDNA derived from said mRNA.

29. The method of claim 1 wherein gene expression is determined by measuring the amount of peptide or polypeptide encoded by the gene in the sample.

30. The method of claim 9 wherein the amount of peptide or polypeptide is determined using a specific binding molecule.

31. The method of claim 1 wherein the subject is human.

32. A method for determining the likelihood that a subject having a viral infection of the liver will be responsive to antiviral therapy that includes stimulation of Interferon (IFN) activity, the method comprising, examining a sample from the subject to identify the subcellular location of STAT1, wherein said subject is likely to be non-responsive to said antiviral therapy if the majority of cells have nuclear localised STAT1.

33. A kit for performing the method of claim 1 comprising:
(i) means for analysing in a sample from a subject the expression of at least one gene from each of the groups of genes listed in claim 1; and, optionally,
(ii) means for comparing expression of the genes in the sample to expression of the same genes in a control sample, wherein an increase in the level of expression of the genes in the sample from the subject before antiviral therapy relative to the level of expression of the same genes in the control sample indicates that the subject is not likely to be responsive to said antiviral therapy.

34. The kit of claim 13, further comprising one or more specific binding molecules that can target molecules representative of said gene expression in the sample.

35. The kit of claim 13 wherein said specific binding molecule is an oligonucleotide probe, antibody, or aptamer.
